(19) United States
(12) Patent Application Publication
(10) Pub. No.: US 2004/0197798 A1
(43) Pub. Date: Oct. 7, 2004

(54) Manipulation and detection of protein phosphatase 2C - PP2Calpha - expression in tumor cells for cancer therapy, prevention and detection

(75) Inventor: Sara Lavi, Kyriat Ono (IL)

Correspondence Address: John P. White, Cooper & Dunham LLP, 1185 Avenue of the Americas, New York, NY, 10036, US

(73) Assignee: Ramot University Authority for Applied Research and Industrial Development Ltd.

(21) Appl. No.: 10/678,477

(22) Filed: Oct. 3, 2003

Related U.S. Application Data
(62) Division of application No. 09/029,479, filed on Oct. 21, 1998, now abandoned.
Application No. 09/029,479 is a 371 of international application No. PCT/IB96/01021, filed on Aug. 30, 1996.
(60) Provisional application No. 60/003,114, filed on Sep. 1, 1995.

(51) Int. Cl. C12Q 1/68

(52) U.S. Cl. 435/6

(57) ABSTRACT

A method of detecting cancer in a patient by detecting alteration of activity of the gene coding for human type protein phosphatase 2C (PP2Cα and PP2Cβ) and genetic polymorphisms thereof in a specimen isolated from the patient is disclosed. The invention further provides a method of treating cancer including the steps of first determining the type of cancer and cells expressing the cancer and then preparing a vector which will specifically target the cancer cells and can include regulatory elements to control the expressibility of PP2Cα. The vector is then administered to the patient. Alternatively an antisense vector can be prepared.

### BACKGROUND OF THE INVENTION

1. Technical Field

The present invention relates to detection and methods of treating cancer by utilizing the gene human type protein phosphatase 2C (PP2Cα and PP2Cβ) and gene products thereof and kits for the practice of the invention; preparing native and transgenic organisms in which the gene products encoded by the human PP2Cα gene or its homolog in other species are produced, or the expression of the native PP2Cα gene is modified or knocked out.

2. Background Art

Transformed or malignant cells pose a severe health threat. If the transformed phenotype can be reversed the cancerous cells can be controlled providing a treatment. In reversal the cells can loose their neoplastic phenotype thereby reinstating normal cellular growth and/or differentiation, or there is growth arrest or there can be activation of programmed cell death—apoptosis pathway. It would be useful to provide therapeutic measures which can reverse the transformed phenotype by instituting any of these reversal means by therapeutic measures. Further, early identification of transformation events would also prove useful, since therapy could be initiated sooner.

Genes have now been identified that are involved in transformation such as Ras, Fos PDGF, erb-B, erb-B2, RET, c-myc, Bci-2, APC, NF-1, RB, p53, etc. The genes fall into two broad categories proto-oncogenes and tumor suppressor genes. Proto-oncogenes code for proteins that stimulate cell division and when mutated (oncogenes) cause stimulatory proteins to be overactive with the result that cells over proliferate. Tumor suppressor genes code for proteins that suppress cell division. Mutations and/or aberrant regulation can cause these proteins to be inactivated thereby rendering the cells without proliferation restraint. Additionally, E2F and p53 and others can act as both oncogene and tumor suppressor gene when improperly expressed. Among the oncogenes and tumor suppressor genes are motifs which act as transcription factors and as protein kinase. The identification of these specific genes have disclosed some of how the cell life cycle progresses.

Gene amplification is one of the distinct abnormalities associated with malignant cells and transformed cell lines [see generally “Gene Amplification in Mammalian Cells, A comprehensive Guide. edited by R. E. Hellems, Marcel Dekker, Inc. for a review of amplification.] This phenomenon is part of the genetic instability characterizing neoplastic cells and occurs rarely in normal cells. Some oncogenes and tumor suppressor genes have been shown to be amplified such as Ras, Erb, p53 etc.

Phosphorylation of structural and regulatory proteins including oncogenes and tumor suppressor genes is a major intracellular control mechanism in eukaryotes [Wera and Hemmings, 1995; Cohen, 1989]. Protein phosphorylation and dephosphorylation is part of the regulatory cycle for signal transduction, cell cycle progression and transcriptional control. Protein kinases and protein phosphatases both have roles in the phosphorylation—dephosphorylation cycle, respectively. Mutations in the genes coding for these proteins can lead to failure of protein phosphorylation. For example, in yeast, mutations of a type 2C protein phosphatases lead to a defect in osmoregulation [Shiozaki and Russell, 1995].

pp2c is a protein serine/threonine phosphatase [Cohen 1989]. The pp2c family consists of two cytoplasmic isoenzymes in mammalian tissues [McGowan and Cohen, 1987] and at least three pp2c-like enzymes in yeast show the same enzymatic and biochemical properties. The two mammalian isoenzymes are monomers but differ slightly in molecular mass (44 KDa and 42 KDa) and are designated pp2cα and pp2cβ. There is conflicting literature as to their function and association of these protein phosphatases with transformed cells [Saadat et al, 1994; Nishikawa, et al, 1995; Lau and Baylink, 1993; Shiozaki et al, 1994; Eden and Cedar, 1994; McGowan and Cohen, 1987; Wenk and Mieskes, 1995]

It would be useful to be able to therapeutically control protein phosphorylation where needed for normal cell function. Additionally, glycosylation following mRNA translation is essential for the functioning of many gene products. Aberrant glycosylation of proteins can interfere with protein function and can result from an altered regulatory pathway. An important mode of control of gene expression is DNA methylation [Eden and Cedar, 1994]. Aberrant methylation of DNA can play a role and lead to improper expression of regulatory proteins controlling cell cycle.

Viruses are very specialized infectious agents that have evolved, in many cases, to elude host defense mechanisms. Adeno associated viruses are members of the family of parvoviruses for which tumor suppressive properties have already been described in 1960 [for review see Rommelaere and Tattersal, 1990]. They are a group of small viruses, with a ssDNA genome of approximately 5000 nucleotides, characterized by identical palindromic termini of 154 bases. The left part of the AAVDA3 genome encodes four multifunctional, overlapping, non-structural proteins (Rep78, Rep68, Rep52 and Rep40) which are translated from differentially spliced mRNA driven by the P5 and P19 promoters (Accession numbers J01901, M12405, M12468, M12469). In the right part of the genome three overlapping capsid polypeptides (VP1-VP3) are encoded from the P40 promoter [Berns, 1990; Leonard and Berns, 1994]. These extremely small DNA viruses are represented in vertebrates by two genera, the autonomously replicating and the helper dependent parvovirus [Siegl et al., 1985].

The helper-dependent adeno-associated viruses (AAV) depend for their replication on coinfecting helper virus (Young and Mayor, 1979a,b), or on conditions of genotoxic stress [Yakobson et al., 1987] and comprise agents infecting humans without apparent disease [Cukor et al., 1984]. Helper viruses are adenoviruses [Atchison et al., 1965], herpes group viruses [Salo and Mayor, 1979] and vaccinia virus [Schlehofer et al., 1986]. The helper viruses share the ability to induce chromosomal damage early in their infection cycle [Schlehofer and zur Hausen, 1982].

Tumor suppressive properties have been found for AAV [for review see Schlehofer, 1994]. It has been shown that the development of tumors induced in rodents by adenoviruses, herpes viruses or by transplantation of cells transformed by these viruses could be inhibited by infecting the animal cells with AAV [Kirschtein et al. 1968; Mayor et al., 1973; de la Maza and Carter, 1981; Ostrove et al., 1981]. The in vivo findings of tumor suppression are paralleled by results showing inhibition of cellular transformation in vitro. This could be shown for cells of different origin (hamster and mouse) transformed by viruses or by activated oncogenes. Compared with controls, cells infected with AAV or transfected with specific AAV DNA sequences displayed decreased focus formation and saturation density indicating inhibition of transformation-associated traits [Casto and Goodheart, 1972; Katz and Carter, 1986; Hermonat, 1989; Hermonat, 1994; Schlehofer et al, 1983; Schlehofer, 1994; Yang et al, 1995; Kleinschmidt et al, 1995].

In addition, there are seroepidemiologic findings in the human population, showing that cancer patients exhibit antibodies to AAV less frequently than matched control individuals. Three independent studies carried out in the USA [Mayor et al., 1976], Belgium [Sprecher-Goldberger et al., 1971] and Germany [Georg-Fries et al., 1984] using different serologic techniques, have found a high prevalence of antibodies to AAV (types 2, 3, and 5) in the normal population contrasting with a relatively low frequency of seropositivity in patients with cancer.

It would be useful to develop therapeutic methods for controlling cell transformation. As the above information indicates, it is possible to reverse cell transformation or to specifically kill the transformed cell. Given the available anti-sense technology, vector delivery technology and the like it would be useful to find human cellular mechanisms that can be controlled to reverse cell transformation with these methods or others as they become known.

### SUMMARY OF THE INVENTION

According to the present invention, a method and kit of detecting cancer in a patient by detecting alterations of the activity of the gene (PP2Cα or PP2Cβ) coding for human type protein phosphatase 2C (pp2cα) and genetic polymorphisms thereof in a specimen isolated from the patient is disclosed.

The invention further provides a method of treating cancer including the steps of first determining the type of cancer and cells expressing the cancer and then preparing a vector which will specifically target the cancer cells and can include regulatory elements to control the expressibility of PP2Cα. The vector is then administered to the patient. Alternatively an antisense vector can be prepared.

The invention further provides a method of treating diseases due to aberrant phosphorylation due to alteration of expression of PP2Cα by controlling PP2Cα expression.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1A-B are graphs of a FACS analysis of CO60 and two AAV/neo cell lines 913 and 916 as prepared for cell cycle analysis. 24 hours after seeding the cell were trypsinized and washed with PBS. The cells were resuspended in 1ml buffer containing 0.1% sodium citrate, 0.1% triton X-100 and 50 μg propidium iodide, and then processed in the FACS.

FIG. 2 is a photograph of a Southern Blot Analysis showing CHINT is associated with AAV integration in different AAV/neo cell lines. Southern blot analysis of different AAV/neo clones, CO60 DNA, digested with BglII, and hybridized with “CHINT” probe. 9-1, 2, 3, 4 and 5 on AAV/neo cell lines. 93R is a revertant that lost the whole chromosome containing the AAV. A6 is a mouse cell line.

FIG. 3 is a schematic representation of the organization of the integrated AAV and the flanking cellular sequences in 9-3 cells. A genomic library was prepared from C9-3 cells using the EMBL-4 lambda phage and scored for AAV positive clones. A clone of 13 Kb-λSL9-1 was isolated and later subcloned to a blue-script vector. Plasmids pSL9-11 (13 Kb), pSL9-8 (10 Kb) and pSL9-6 (3 Kb) were obtained as indicated in the figure.

FIG. 4A-B wherein (A) is a photograph of a Southern Blot Analysis showing AAV is adjacent to the gene coding to PP2Cα in 9-3 cells, The Southern blot analysis of genomic DNA, from CO60 and 9-3 cells digested by EcoRI, or XbaI was hybridized sequentially with the following probes: 1) AAV; 2) CHINT; and 3) Rat PP2Cα probes. The CHINT and the PP2Cα sequences are adjacent (4 Kb EcoRI fragment). The AAV CHINT and PP2Cα are in a close proximity in 9-3 cells (the 5.6 Kb XbaI fragment). (B) pSL9-6 is adjacent to PP2Cα in the wild type Chinese hamster cells. BamHI digested DNA from the Chinese hamster neo cells was hybridized to pSL9-6 and PP2Cα probes. A common fragment of ˜8.5 Kb appeared in all cell lines including CO60. The same fragment hybridized also to CHINT probe (data not shown).

FIG. 5A-C are photographs of a Southern blot analysis of DA3 (lane 8) and DA3J1-DA3J7 cells lines (lanes 1-7). Genomic DNA was digested with BglII. The blots were hybridized sequentially with an AAV/neo JDT277, pSL9-6 and PP2Cα PCR probes. A 4 Kb fragment hybridized to the AAV probe and pSL9-6 probe in J3 (lane 3), J4 (lane 4) and J6 (lane 6). A fragment smaller than 4 Kb hybridized to both AAV and PP2Cα probe in J1 (lane 1), J2 (lane 2), J5 (lane 5) and J6 (lane 6).

FIG. 6 is a photograph which shows the alteration in PP2Cα mRNA in response to carcinogen treatment. Forty μg of total RNA were isolated from CO60 and C9-3 cells 48 hours after treatment with MNNG (7.5 μ/ml and 2.5 μg/ml respectively), and from untreated cells and fractionated on a denaturing gel (1.2% agarose/6.6% formaldehyde gel). The gel was blotted and hybridized consecutively with ³²P-labeled rat PP2Cα cDNA (A) pSL9-1^{DNA} (B) and rRNA cDNA (C)

FIG. 7 is a photograph which shows gel electrophoresis anaylsis after 25, 30 and 35 PCR cycles. The oligo dT-primed cDNA obtained from colorectal tumor No. 6 (T6), or from its adjacent nontumorous mucosa (N6), were subjected to PCR reactions using the specific PP2Cα and the β-actin sense and anti-sense primers. The 25 cycle PCR cycle for PP2Cα cDNA in the normal and tumor tissues is not shown since a visible product was not found.

FIG. 8 is a photograph which shows gel electrophoresis anaylsis after 25, 30, 30 and 35 PCR cycles of aliquots of the oligo dT-primed cDNA obtained from CHE cell line, or from its adjacent transformed cell line (CO60), subjected to PCR reactions using the specific PP2Cα and the β-actin sense and anti-sense primers.

FIG. 9A-B are schematic representations of plasmids that contain PP2Cα cDNA in the (A) sense orientation (pYM001) and in the (B) antisense orientation (pYM002).

FIG. 10 is a photograph which shows gel electrophoresis anaylsis of immunoprecipitation of liver extracts with a panel of monoclonal antibodies raised against pp2cα; 1D5, 2A3, 9F4, 9F1, are monoclonal antibodies used to precipitate pp2cα from liver extract; 801 and 351 are rabbit polyclonal antibodies used for detection after immunoblotting.

FIG. 11 is a schematic representation of a genomic λ100 clone containing the first translated exon of PP2Cα. The phage was cloned from a CHO library. The sequenced regions are indicated by cross hatching (SEQ ID Nos:15 and 16).

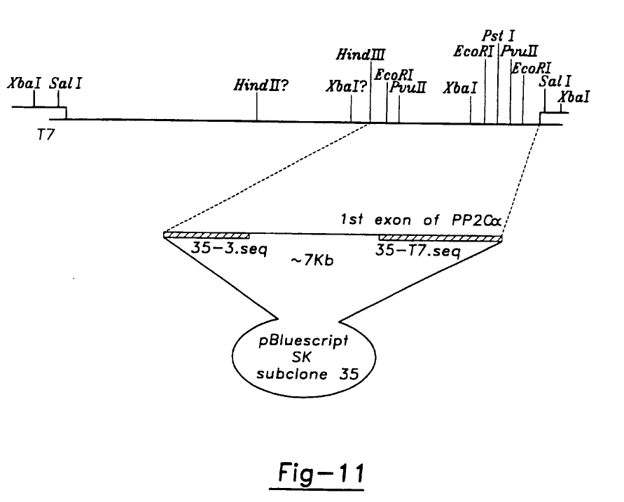

FIG. 12 is a photograph which shows gel electrophoresis wherein lane 1: Cotransfection with pSK1 and pAV2; lane 2: Transfection with the SV40 plasmid pSK1 SV40 replicates; lane 3: Cotransfection of pSVK1 and a plasmid harboring 140 bp from the AAV genome nucleotide 125-263; lane 4: Cotransfection of pSVK1 with pSL9-6.

FIG. 13 is a photograph of a Northern blot wherein RNA from various mouse tissues is hybridized with PP2Cα cDNA demonstrating that there are several mRNAs of different sizes ranging from less than 2 kb to higher than 5.0 kb. RNA was extracted from ovary (O), Testis (T), Kidney (K), Liver (L), Muscle (M), Heart (H), Lung (Lu) and Brain (B).

### DETAILED DESCRIPTION OF THE PREFERRED EMBODIMENT

The present invention discloses a method of detecting cancer in a patient by detecting alterations in gene activity of the gene (PP2Cα) coding for human type protein phosphatase 2C (pp2cα) and genetic polymorphisms thereof in a specimen isolated from the patient. The gene activity of the patient is compared to that of normal controls. Alterations in activity can be a down-regulation of the gene activity or conversely an up-regulation resulting in changes in phosphorylation. Further, alterations can result in aberrant function or absence of the gene product and in a change in distribution of the gene product within the cell itself.

Polymorphisms are variants in the gene sequence. They can be sequence shifts found between different ethnic and geographic locations which, while having a different sequence, produce functionally equivalent gene products, isoforms. Polymorphisms also encompass variations which can be classified as alleles and/or mutations which can produce gene products which may have an altered function. Polymorphisms also encompass variations which can be classified as alleles and/or mutations which either produce no gene product, an inactive gene product or increased levels of gene product. Polymorphisms as used herein can also encompass variations which are due to differences in DNA methylation in control and coding regions. Further, the term is also used interchangeably with allele as appropriate.

Cancer is defined as transformed or malignant cells, i.e. cells undergoing uncontrolled growth and spread (see generally, Scientific American September, 1996 for a review).

In general, it is found in cell transformation as shown in the Examples herein below (Examples 4, 5) that the activity/expression of PP2Cα is reduced compared to that of normal controls as determined by a reduction in the amount of gene product in the cell. However, it is contemplated by the present invention that increased activity results in changes in protein activity such that there are changes in cell function.

Further it is recognized by the present invention that there is more than on form of the gene product of PP2Cα and that one may be reduced or altered in cells while another specific form of PP2Cα will be elevated or more prominent compared to normal controls. The cells can be any cell type that shows alteration in PP2Cα activity in a disease state. Further, a second gene may be controlled by the alteration in the activity of PP2Cα such that their products are elevated or reduced and can be monitored by the method of the present invention. New transcripts, absence of transcripts or alterations in the protein coded by these transcripts are monitored.

Further, the present invention recognizes that pp2cα is itself phosphorylated as it has several phosphorylation sites including tyrosine, serine and thyronine and that failure to phosphorylate it properly will cause malfunction of the pp2cα protein. Further, pp2cα also dephosphorylates itself. A failure in its autophosphorylation will have effects on cell cycle regulation.

Samples can be biopsied material from suspected precancerous lesions or any tissue or bodily fluid which can be assayed for PP2Cα activity or gene product as described herein. Bodily fluids such as blood, urine, cerebrospinal fluid and saliva can be examined as is appropriate.

In an embodiment the detection of PP2Cα activity is by assaying the specimen for mRNA complementary to PP2Cα DNA including polymorphisms thereof with an assay selected from the group consisting of in situ hybridization, Northern blotting and reverse transcriptase—polymerase chain reaction.

In an alternative method, the detecting of PP2Cα activity and cellular distribution is by assaying the specimen for a PP2Cα gene product including polymorphisms and peptide fragments thereof with an assay selected from the group consisting immunchistochemical and immunocytochemical staining, ELISA, RIA, immunoblots, immunoprecipitation, Western blotting, functional assays for activity of gene product, assays for phosphorylation patterns and protein truncation test. Target proteins which are dephosphorylated by pp2cα can have different size characteristics on PAGE and different isoelectric points as well as changes in function such as their ability to interact with other proteins, RNA, DNA and other cellular components.

Further, if chromosomal abnormalities are associated with altered PP2Cα these are screened for using standard methods known in the art.

In addition to changes in the location and amount of the gene product in the cell itself as shown in the Examples herein below, the method of the present invention screens for the gene product in bodily fluids. With alteration in gene function the level of gene product in the bodily fluid is affected as for example more can be released from the cell if glycosylation or signal sequences are affected. Incomplete protein fragments may result from interrupted translation which are then released from the cell and are monitored.

Further, the present invention recognizes alternately spliced forms of the mRNA for pp2cα giving rise to different sizes and/or function in different tissues and assays are designed to recognize the alternately spliced forms in the appropriate tissues.

The identification of alterations in the gene product in a specific bodily fluid will indicate the source/location of a tumor. For example, with a tumor in the central nervous system, the gene product would be found in the cerebrospinal fluid. Similarly the location of other tumors or other diseases would determine which bodily fluids to screen and the converse as would be known to those skilled in the art.

The present invention also provides for a kit for detecting PP2Cα activity and/or alteration either at the mRNA level or gene product level. The kit includes molecular probes for mRNA for PP2Cα mRNA and detection means for detecting the molecular probe and thereby the mRNA. Alternatively, or in addition, the kit can contain probes for detecting the PP2Cα gene product. The detecting means are in general are antibodies with high specificity for the gene product or agents which mimic natural proteins which bind to the PP2Cα gene product other agents as known in the art may also be used. The antibodies are made as described herein below and in Example 3, which specifically recognize the PP2Cα or PP2Cβ gene products (including on the cell surface) including polymorphisms thereof, and detection means for detecting the binding of the antibody thereby indicating the presence of the gene product and also distinguishing one from the other.

Where appropriate, the kits can also contain antibodies directed against secondary gene products that are affected by the alteration in function of the PP2Cα gene.

The present invention discloses a method of detecting cancer in a patient by detecting altered levels of PP2Cβ gene activity compared to normal patients in a specimen isolated from a patient.

The present invention also provides for a kit for detecting PP2Cβ activity. The kit includes molecular probes for mRNA for PP2Cα polymorphisms thereof and detection means for detecting the molecular probe and thereby the mRNA or antibodies or other means of identifying alterations in the level of the gene product over normal controls as described herein.

The present invention provides an antibody, either polyclonal or monoclonal, which specifically binds to a polypeptide/protein encoded by the PP2Cα gene as described in Example 3 herein below. The antibodies of the present invention are used in identifying the gene product of PP2Cα and PP2Cβ. The present invention provides monoclonal and polyclonal antibodies raised against recombinantly produced PP2Cα, NDDTDSASTD (SEQ ID No:1), YKNDDTDSTSTDDMW (SEQ ID No:2), recombinantly produced pp2cβ and PNKDNDGGA (SEQ ID No:3).

The present invention also provides for isolated and purified peptides NDDTDSASTD (SEQ ID No:1), YKNDDTDSTSTDDMW (SEQ ID No:2) and PNKDNDGGA (SEQ ID No:3). The peptides can be produced recombinantly.

The invention further provides antibodies that will recognize the special structures at the 5′UTR or the RNA-proteins complexes responsible for the controlled expression of PP2Cα. Antibody which recognizes specifically the special RNA structures is also provided.

In general in preparing the antibody, either the entire pp2cα protein or peptide sequences thereof can be used as an immunogen as well as polymorphisms thereof. Further, anti-idiotypic antibodies can be made against these antibodies. The antibodies may be either monoclonal or polyclonal. Conveniently, the antibodies may be prepared against a synthetic peptide based on the sequence, or prepared recombinantly by cloning techniques or the natural gene product and/or portions thereof may be isolated and used as the immunogen. Such proteins or peptides can be used to produce antibodies by standard antibody production technology well known to those skilled in the art as described generally in Harlow and Lane, Antibodies: A Laboratory Manual, Cold Spring Harbor Laboratory Press, Cold Spring Harbor, N.Y., 1988.

For producing polyclonal antibodies a host, such as a rabbit or goat, is immunized with the protein or peptide, generally with an adjuvant and, if necessary, coupled to a carrier; antibodies to the protein are collected from the sera.

For producing monoclonal antibodies, the technique involves hyperimmunization of an appropriate donor, generally a mouse, with the protein or peptide fragment and isolation of splenic antibody producing cells. These cells are fused to a cell having immortality, such as a myeloma cell, to provide a fused cell hybrid which has immortality and secretes the required antibody. The cells are then cultured, in bulk, and the monoclonal antibodies harvested from the culture media for use.

The antibody can be bound to a solid support substrate or conjugated with a detectable moiety or be both bound and conjugated as is well known in the art. (For a general discussion of conjugation of fluorescent or enzymatic moieties see Johnstone and Thorpe, Immunochemistry in Practice, Blackwell Scientific Publications, Oxford, 1982.) The binding of antibodies to a solid support substrate is also well known in the art. (see for a general discussion Harlow and Lane Antibodies: A Laboratory Manual, Cold Spring Harbor Laboratory Publications, New York, 1988) The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination. Additionally, toxins can be coupled to the antibody for targeted delivery.

The present invention also provides for transgenic human PP2Cα gene and polymorphic PP2Cα gene, animal and cellular (cell lines) models as well as for knockout PP2Cα models. These models are constructed using standard methods known in the art and as set forth in U.S. Pat. Nos. 5,387,742, 5,360,735, 5,347,075, 5,298,422, 5,288,846, 5,221,778, 5,175,385, 5,175,384, 5,175,383, 4,736,866 as well as Burke and Olson, [1991], Capecchi, [1989], Davies et al., [1992], Dickinson et al., [1993], Huxley et al., [1991], Jakobovits et al., [1993], Lamb et al., [1993], Rothstein, [1991], Schedl et al., [1993], Strauss et al., [1993). Further, patent applications WO 94/23049, WO 93/14200, WO 94/06908, WO 94/28123 also provide information.

The present invention provides vectors comprising an expression control sequence operatively linked to the nucleic acid sequence of the PP2Cα gene and portions thereof as well as polymorphic sequences thereof (see Examples herein below). The present invention further provides host cells, selected from suitable eucaryotic and procaryotic cells, which are transformed with these vectors.

The vectors can be introduced into cells or tissues by any one of a variety of known methods within the art. Such methods can be found generally described in Sambrook et al., Molecular Cloning: A Laboratory Manual, Cold Springs Harbor Laboratory, New York (1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995) and Gilboa, et al (1986) and include, for example, stable or transient transfection, lipofection, electroporation and infection with recombinant viral vectors. Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands [Solderling, 1993] to alter target specificity through receptor mediated events.

More specifically, such vectors are known or can be constructed by those skilled in the art and should contain all expression elements necessary to achieve the desired transcription of the sequences. Other beneficial characteristics can also be contained within the vectors such as mechanisms for recovery of the nucleic acids in a different form. Phagemids are a specific example of such beneficial vectors because they can be used either as plasmids or as bacteriophage vectors. Examples (see Example herein below) of other vectors include viruses such as bacteriophages, baculoviruses and retroviruses, DNA viruses, cosmids, plasmids, liposomes and other recombination vectors. The vectors can also contain elements for use in either procaryotic or eucaryotic host systems. One of ordinary skill in the art will know which host systems are compatible with a particular vector.

Recombinant methods known in the art can also be used to achieve the sense, antisense or triplex inhibition of a target nucleic acid. For example, vectors containing antisense nucleic acids can be employed to express protein or antisense message to reduce the expression of the target nucleic acid and therefore its activity. Additionally, ribozymes can be generated and used to “knock-out” the mRNA expression of the gene [Cech, 1986; Cech, 1990; Hampel et al, 1993; Sullivan, 1994].

A specific example of DNA viral vector for introducing and expressing recombinant sequences is the adenovirus derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur. Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways and in combination with a suitable pharmaceutical carrier. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration, especially in the case of neuro-degenerative diseases. Following injection, the viral vectors will circulate until they recognize host cells with the appropriate target specificity for infection.

An alternate mode of administration of a PP2Cα vector can be by direct inoculation locally at the site of the disease or pathological condition or by inoculation into the vascular system supplying the tumor with nutrients. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by one skilled within the art.

In a preferred embodiment, a virus vector based on modified AAV is used. AAV has been shown to integrate into the human genome in chromosome 19q13.3. Alteration of the AAV genome in a mode that will allow it to integrate in a site specific manner into the PP2Cα regulatory region is used. (see Example 7)

The invention further provides a method of treating cancer including the steps of first determining the type of cancer and cells expressing the cancer and then preparing a vector as described herein above which will specifically target the cancer cells and includes regulatory elements to control the expressibility of PP2Cα. The vector is then administered to the patient and can include a suitable pharmaceutical carrier which will not affect bioactivity of the vector. Alternatively an antisense vector can be prepared and used to control the expression of PP2Cα.

pp2c is a protein serine/threonine phosphatase [Cohen 1989]. It is unique among phosphatases since it requires magnesium and is not sensitive to certain phosphatase inhibitors such as okadaic acid [Cohen 1991]. The pp2c family consists of two cytoplasmic isoenzymes in mammalian tissues [McGowan and Cohen, 1987] and at least three pp2c-like enzymes in yeast show the same enzymatic and biochemical properties. The two mammalian isoenzymes are monomers but differ slightly in molecular mass (44 KDa and 42 KDa) and are designated pp2cα and pp2cβ. There is 70% homology between the α and β isoforms. At the carboxy terminal of the pp2cα there is a fifteen amino acid sequence which differs from pp2cβ. In humans the sequence is YKNDDTDSTSTDDMW (SEQ ID No:2).

A 106 kb cosmid coding for pp2cα and additional proteins FosB and ERCCI has been sequenced [Martin-Gallardo et al., 1992] (GENBANK accession number: M89651). Further, the cDNA sequences of PP2Cα in humans is known [Mann et al, 1992]. However, attempts to align the 5′UTR of the cDNA with the genomic sequences were not successful.

A hypothesis for the above observations can be made, but it is not to be construed as limiting the present invention to this one mode. Applicants propose that the UTR consists of several small exons with large introns and propose that PP2Cα and FosB have a common regulatory region (ERCCI may also share the regulatory region). Alternatively, it is possible that PP2Cα is a very large gene and that the 5′ end and the control region do not reside within the 106 kb cosmid in a region located 5′ to the 106 kb cosmid. As a further alternative, the region of 9 kb from the cosmid was not sequenced due to the high G/C content and it may contain the 5′UTR region and the promoter.

In a preferred embodiment the AAV virus and/or CHINT or other regulatory sequences related to the PP2Cα gene are used in the vector, particularly those which are used to treat patients. CHINT is a cellular sequence which was recombined into the AAV in 9-3 cells; the sequence is set forth in Table 5 (int.li; SEQ ID No:19). The vector can either integrate into the regulatory control of PP2Cα and alter its expression in the same way as AAV alters cells into which it integrates as it is an oncosuppressive virus. This thereby reinstates normal cellular growth, or there is growth arrest or there can be activation of programmed cell death—apoptosis pathway depending on the cell type, type of cancer, stage of differentiation, and other factors as known to those skilled in the art. [Schlehofer, 1994]

There are several elements in AAV and/or CHINT or in the PP2Cα regulatory elements that can be used to control PP2Cα expression taking advantage of the following observations:

1. PP2Cα has a very long 5′ and 3′ UTR (they are larger than the coding capacity). Specific folding of the RNA and interaction with specific sets of proteins might effect its expression dramatically. At certain stages there might different modes of folding and these different proteins may interact with the RNA and alter its expression.

2. The CHINT sequences involved in the integration have very interesting motifs which might be used for the site specific integration. Moreover, applicant has data demonstrating that specific AAV sequences adjacent to the AAV integration site are responsible for the suppression of DNA amplification. These sequences can be used in vectors as a therapeutic and are described herein below as silencer (SEQ ID No:13) and mini-silencer (SEQ ID No:14).

The invention further provides a method of treating cancer by using an AAV based vector or other vector for cancer treatment that only functions specifically in cells in which PP2Cα is improperly activated. The vector is administered to those who have been diagnosed with a tumor as is known to those skilled in the art. The AAV vector (or other regulatory factor as disclosed herein) in one embodiment is under the control of a promotor, rep, that is expressed in transformed cells. The integrated vector will control PP2Cα expression in the cell reversing transformation as shown in the Examples. Further, the vector will be targeted to the cell type that has been transformed.

Further, since the gene product of PP2Cα is expressed on the cell surface, antibody directed against the gene product can activate/inactivate the expression of the gene via the signal transduction pathway. Therefore antibodies can be used therapeutically to treat patients in which the PP2Cα gene needs to be re-regulated. The use of Fab fragments and other means known in the art can be used to insure that the antibodies upon administration to a patient do not have secondary unwanted effects. Alternatively, a ligand or other molecule which can specifically bind to the PP2Cα gene product can be used. The present invention therefore provides a method of binding the gene product of PP2Cα expressed on the surface of a cell to induce signal transduction thereby suppressing the transformed phenotype.

The invention further provides a method of treating diseases due to aberrant phosphorylation due to alteration of expression of PP2Cα by controlling PP2Cα expression. Such disease can be neurologic. For example, behavioral changes could be associated with aberrant phosphorylation. As shown by Brown et al, 1996 mutations in fosB can lead to behavioral changes. As discussed herein above, fosB and PP2Cα are on the 106 kD cosmid. There is some indication that they may be co-regulated. Therefore aberrant expression of PP2Cα can be expressed as behavioral changes. Further, the levels of PP2Cα activity are extremely high in cardiac and kidney tissues compared to other tissues. Therefore alterations in PP2Cα activity will be reflected in these tissues.

The present invention provides a method of suppressing gene amplification by interrupting the binding or action of DNA polymerase a primase and RNA polymerase II with the gene product of PP2Cα by preparing an antisense vector which will specifically target the binding region of DNA polymerase a primase and RNA polymerase II to the PP2Cα gene product and delivering the vector to the cells as based on the observations set forth in Example 9. Applicants have observed that in tumor cells pp2cα binds to the CTD domain of RNA polymerase II. Therefore alternatively, delivery of a peptide with the CTD domain can be used via competitive binding strategies to control the binding leading to gene amplification.

To investigate the role of AAV in tumor suppression an AAV/neo virus JDT277 was introduced into SV40 transformed Chinese hamster (CO60 and OD4 cells) and mouse mammary tumor cells (DA3). CO60 is a cell line of SV40 transformed Chinese hamster embryo cell lines [Lavi, 1981]. The OD cell line was established by transfection of Chinese hamster embryonic cells with origin deleted SV40 DNA [Lavi, 1985]. The mouse DA3 cell line was derived from mammary tumors syngeneic to BALB/c mice [Sotomayor et al, 1991]. JDT277 virus contains the portion of the AAV2 genome, which encodes the viral Rep proteins, the AAV terminal inverted repeats (TIRs) and the prokaryotic neomycin phosphotransferase gene (neo), conferring resistance to G418. The neo gene was inserted at nucleotide 1882, resulting in carboxy terminus truncated Rep proteins. The truncation of the rep proteins does not affect the ability of the AAV/neo virus to replicate in Adenovirus coinfected human cells.

Single colonies were isolated and amplified by serial passages in the presence of G418. The resistant cells were designated 9-1 to 9-5 for cells derived from CO60 cells and A20-A29 for cells derived from OD4 cells. The cell lines derived from the mouse cells DA3 were designated J1-J15.

Alterations of the Transformed Phenotype:

Following AAV integration the cells lost several of their transformed characteristics.

1. Suppression of SV40 DNA amplification. (Example 7)

A characteristic trait of tumor cells is their capability to amplify DNA. CO60 cells are used as a model system to study gene amplification and SV40 amplification can be induced in the cells as a results of treatment with carcinogens [Lavi, 1981; Aladjem and Lavi, 1992]. Following AAV/neo virus integration, the cells were incapable to amplify SV40. Most AAV/neo cell lines derived from CO60 cells lost their capability to amplify SV40 upon treatment with carcinogen in contrast to the parental CO60 cells [Tal Burstyn, 1993]. Extracts from AAV/neo cells derived from both OD4 and CO60 cells lost their capability to amplify SV40 in vitro [Winocour et al., 1992; Tal Burstyn, 1993].

2. The cells harboring the integrated SV40 became highly sensitive to treatment with UV or MNNG. [Winocour et al., 1992].

3. The transformed cells lost their capability to grow in soft agar, a characteristic typical to transformed cells.

4. The cells displayed apoptotic phenotype as measured by

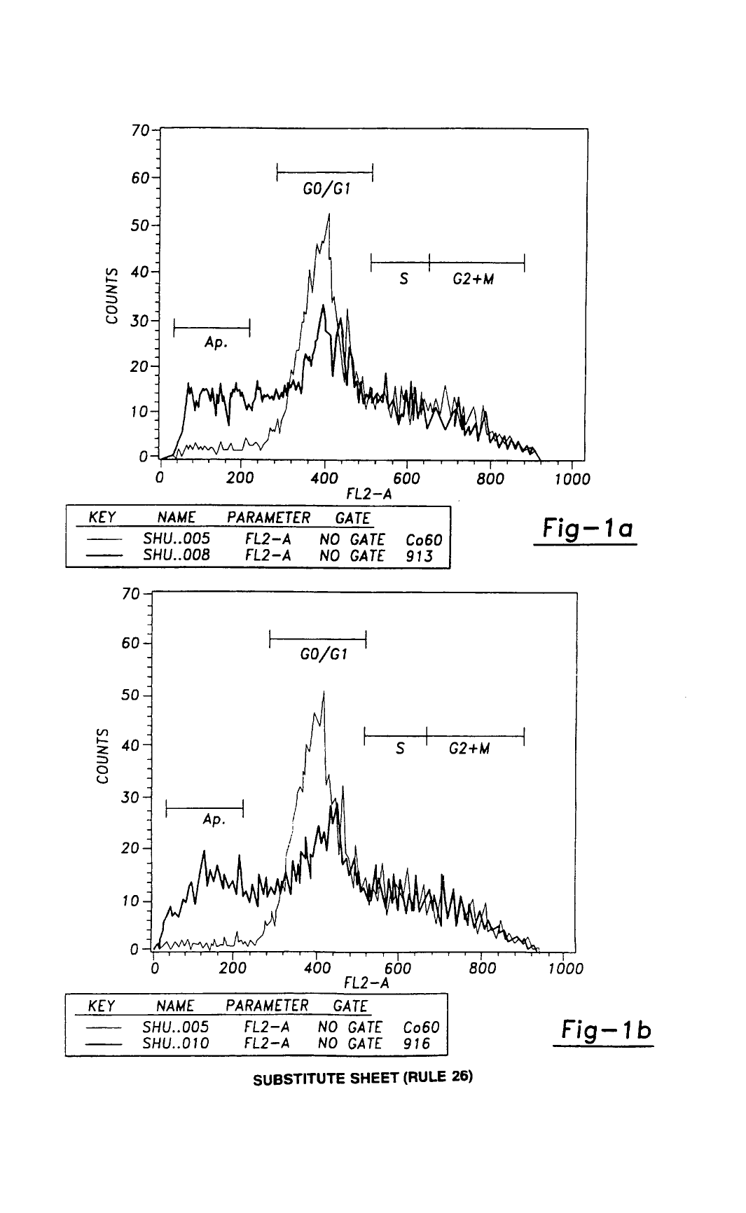

a) the cell cycle pattern was altered and apoptotoic cells appeared spontaneously in the AAV/neo cells and the level was enhanced following treatment with DNA damaging agents. (FIG. 1A, Table 3A and FIG. 1B, Table 3B),

b) the condensation and fragmentation of the chromatin and cytoplasm. Condensation and fragmentation of chromatin was monitored by staining with acridine orange. Ethidium bromide did not stain these cells.

Acridine orange is taken up by both live and dead cells creating fluorescent-green signals, whereas ethidium bromide is only taken up by nonviable cells and gives bright red fluorescence. This double staining system provides a means to distinguish between dead and living cells, and cells that underwent apoptosis before they lost their membrane integrity. Both normal or apoptotic nuclei in living cells fluoresce bright green. In striking contrast, normal and apoptotic nuclei in dead cells fluoresce bright red.

A substantiated fraction of the cells displayed apoptotic nuclei showing condensed chromatin upon staining with acridine orange. In addition, the cells displayed a strong shrinkage of the cytoplasm. Often the nuclei were disrupted into a multitude of micronuclei. These cells underwent apoptosis without losing their membrane integrity. EtBr did not penetrate into these cells, thus the cells were still alive. A large amount of living apoptotic nuclei were found in the treated AAV positive cells compared to a considerably lower percentage in the treated (7.5 μg/ml MNNG) and control CO60 cells. The same pattern of staining repeated in all the AAV/neo cell lines. Hence, this apoptotic phenotype was a common feature to all the AAV/neo cells.

c) by the breaks in the chromosomal DNA:

Programmed cell death was shown to be associated with DNA fragmentation. This approach to detect apoptosis was based on the specific binding of terminal deoxynucleotidyl transferase (TdT) to 3′-OH ends of DNA, ensuing a synthesis of polydeoxynucleotide polymer [Gavrieli et al., 1992]. By adding biotinylated deoxyuridine to fixed cells, TDT was used to incorporate these nucleotides at sites of DNA breaks. The signal was amplified by FITC-Avidin binding enabling identification by fluorescence microscopy. In all AAV/neo cell lines, Applicants could detect a distinct pattern of nuclear staining, directly correlated to the typical degradation of chromatin in apoptotic cells. Since this reaction is specific, only the apopotic nuclei are stained. As a positive control for the efficiency of the technique, Applicants used CO60 nuclei treated with DNase.

C9-2 and C9-3, the AVV positive clones displayed a bright nuclear fluorescence 48 hours after treatment with 2.5 μg/ml MNNG, whereas the controls without any treatment showed only a low background fluorescence. Applicants could see the characteristic degradation of the nuclei into a multitude of small highly fluorescent micronuclei. The fluorescence obtained was similar to the positive control.

Untreated AAV/neo cells and control and treated CO60 (7.5 μg/ml MNNG) did not show any sign of fluorescence. This pattern of nuclear degradation appeared in all AAV/neo cell lines tested (approximately 20), however, the extent of fragmentation varied in the different lines.

Unexpectedly, the revertant cells, designated C9-3-2 and C9-3-12, which were selected on the basis of loss of resistance to G418, and lost their integrated AAV sequences [Burstyn, 1993], still maintained their apoptotic phenotype following treatment with 2.5 μg/ml or 5 μg/ml MNNG.

Further analysis by FACS, Giemsa staining, and electrophoretic separation of the nucleosomal DNA fragments from high molecular weight cellular DNA supported these results.

The Integrated AAV in the AAV/Neo Cells (Examples 6 and 7)

Analysis of cell extract derived from the Chinese hamster A20-A29, 9-1 to 9-5 and the mouse DA3 derivatives for the expression of rep proteins by immunoblotting with anti-Rep antibodies demonstrated that in all cell lines the authentic Rep products were not present. In some cell lines a short protein, probably a truncated protein, reacted with the anti-Rep antibodies [Winocour et. al., 1992].

PCR analysis of most cell lines for the presence of the intact rep promoter region demonstrated that in most cell lines the promoter region of the Rep protein was reorganized as a result of deletions, insertions and rearrangements of the AAV sequences thus eliminating the expression of the authentic Rep proteins.

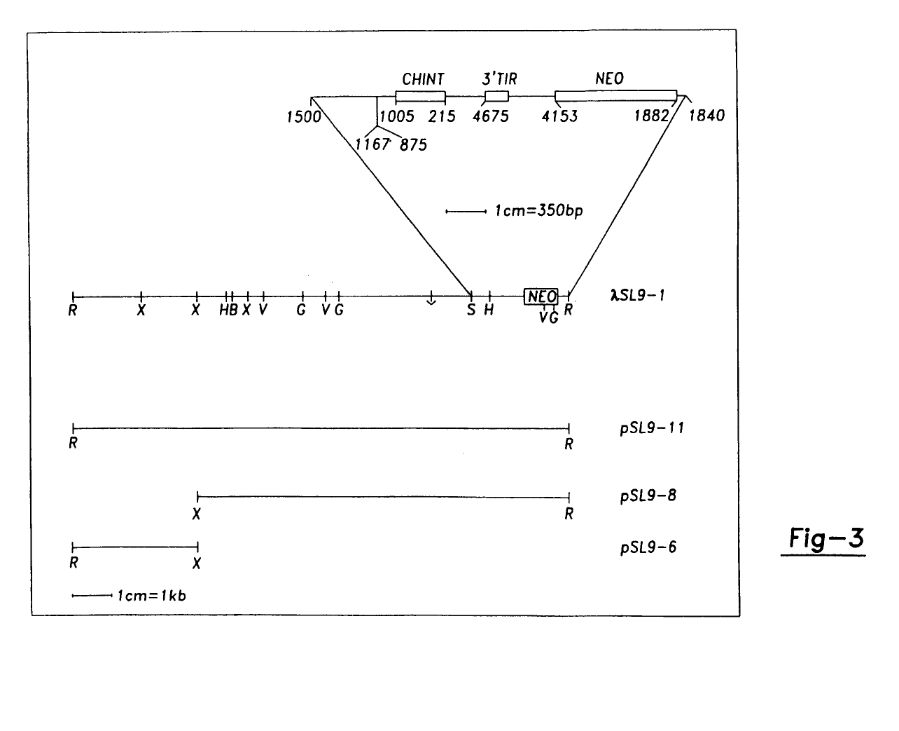

Applicant focused on the analysis of one Chinese hamster cell lines, 9-3, derived from CO60 cells (FIG. 3). The integrated AAV undergoes duplication in this cell line and the chromosome harboring the AAV contains two regions in which AAV is integrated. This duplication of AAV probably resulted from the massive rearrangement which occurred in the Chinese hamster genome following AAV integration. In all Chinese hamster cell lines studied so far by in situ hybridization (6 independent cell lines) the chromosome harboring the integrated AAV was altered and was different in many respects from all the typical Chinese hamster chromosomes, thus the identity of the chromosome could not be established.

The integrated AAV and flanking cellular sequences for 9-3 were cloned into a phage (FIG. 3). As diagramed in FIG. 3 the viral genome underwent several changes. Sequences downstream to the AAV p5 promoter were deleted and replaced by a cellular fragment “CHINT”. In addition, deletions and rearrangements in the 5′ portion of the AAV/neo genome were observed. In contrast, the region coding for the Neo gene and the 3′ end of the viral genome remained intact. (Similar alterations were observed in all AAV/neo Chinese hamster and mouse cell lines tested).

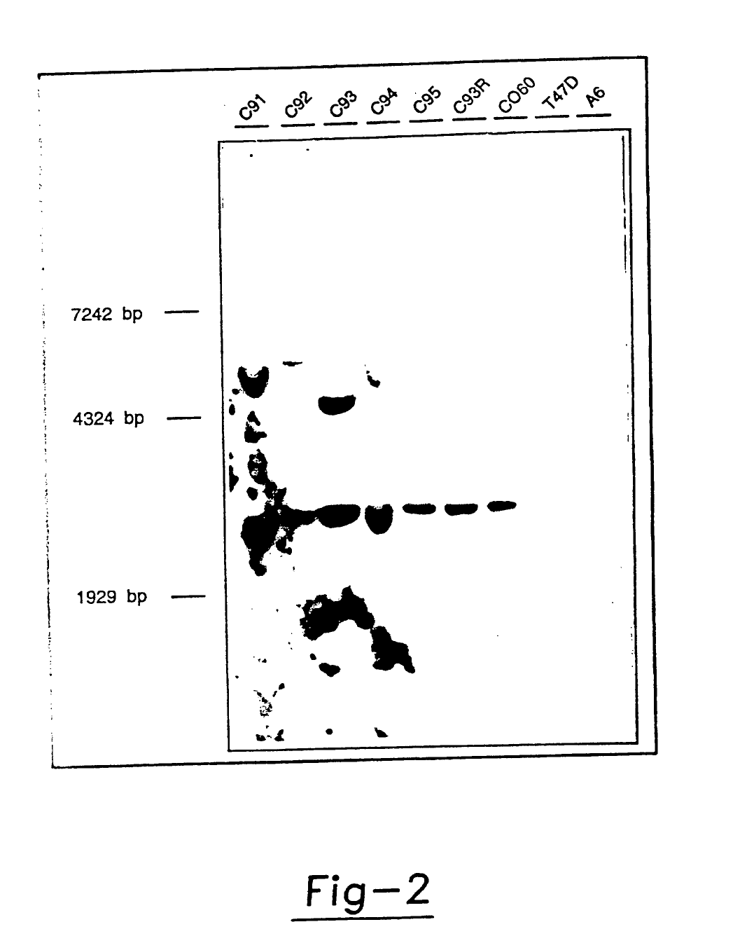

The “CHINT” sequences were used as a probe to analyze AAV integration site in different AAV/neo Chinese hamster clones (FIG. 2). In several AAV/neo clones there was a shift in this fragment suggesting that the size of this fragment was altered upon AAV integration. Some of the shifted bands also hybridized to AAV probe demonstrating that AAV indeed integrated into this region. A probe from the subclonned plasmid pSL9-6 derived from the flanking cellular sequences (FIG. 3) was hybridized to the mouse DA3 AAV/neo cells and in most cases it was associated with AAV integration. These results suggest that the AAV integration site in Chinese hamster might be similar to the one in mouse cells.

Sequence comparison using Genetic Computer Group Inc. software demonstrated that the CHINT sequences are 58.3% homologous to a sequence in the human chromosome 19q13.3. This human sequence is a part of a 106 Kb fragment which was automatically sequenced and analyzed [Martin-Gallardo et al., 1992] (GENBANK accession number: M89651). The region which showed homology to the CHINT sequences was a part of the gene coding for human type protein phosphatase 2C (pp2cα). According to the cDNA sequence of this gene the exact region homologous to CHINT is located upstream to the 5′UTR of PP2Cα (Table 5) (GENBANK accession numbers: human PP2Cα S87759, rabbit PP2Cα S87757).

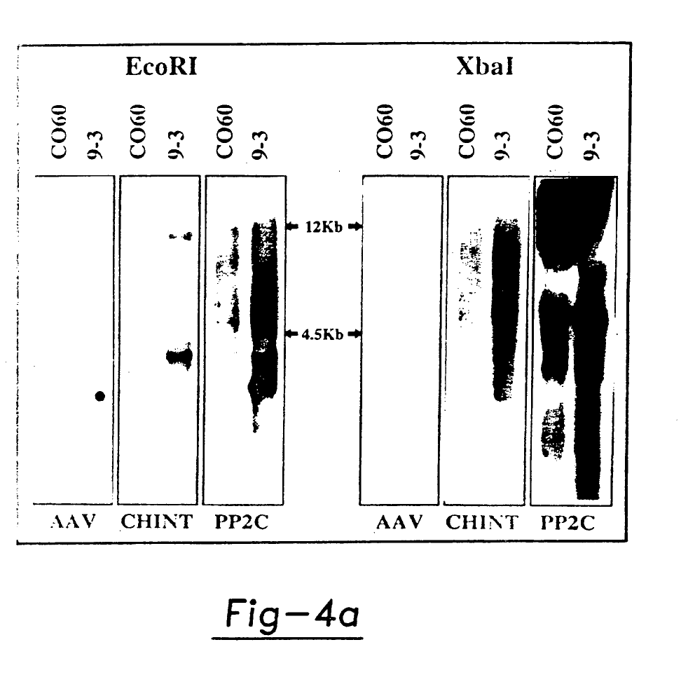

Using a PCR fragment derived by using two primers (Primers 1 and 4, Methods herein below) for PP2Cα, cDNA DNA from 9-3 was probed and a XbaI fragment was found which hybridized to AAV, CHINT and PP2Cα, and an EcoRI fragment was found which hybridized to PP2Cα and CHINT in CO60 DNA. Thus, PP2Cα is indeed localized in very close proximity to CHINT in CO60 cells and is at the integration site in 9-3 cells. In situ hybridization confirmed this conclusion (FIG. 4A).

In both Chinese hamster (CO60 and OD4) and mouse cells (DA3) a portion of the PP2Cα sequences was adjacent to the CHINT hamster sequence present in pSL9-6 (FIG. 3) which was derived from the lambda clone of the AAV integration site. Thus in both the normal Chinese hamster and mouse chromosome PP2Cα is indeed localized at very close proximity, less than 4 Kb from the sequences surrounding the integrated AAV (FIG. 4B).

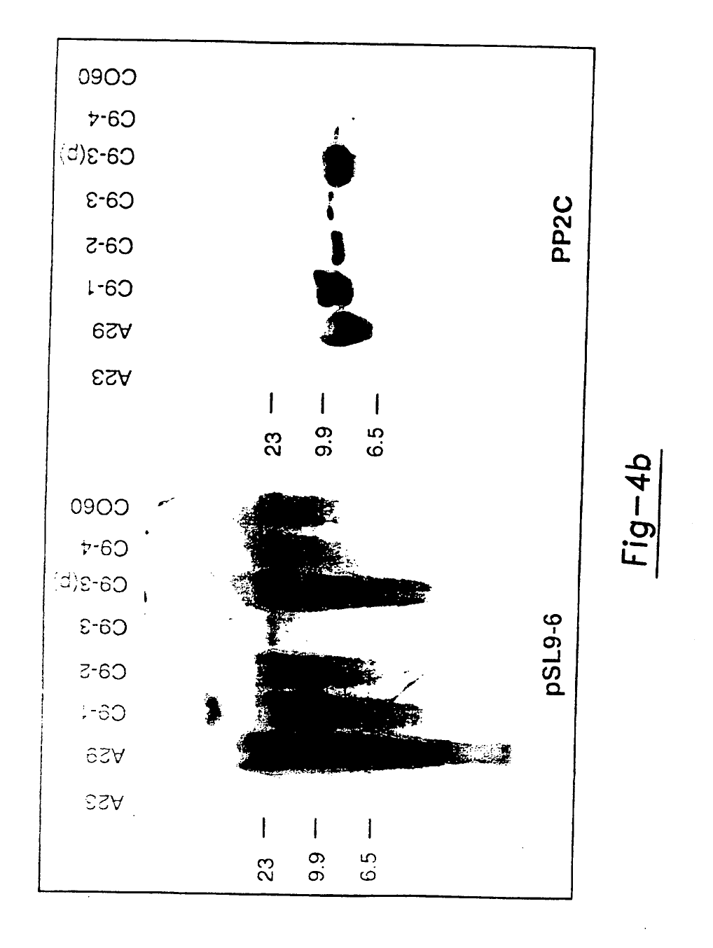

Furthermore, in mouse DA3 cells harboring the integrated AAV genome the AAV sequences were associated with PP2Cα or with fragment 6 or with both (FIG. 5). The integrated AAV was cloned from DA3J7 (λDA37A) and parts of the phage were sequenced as shown in the figure. Homology was found to a region within the integrated AAV in the λSL9-1 plasmid (FIG. 3) and to the human chromosome 19q13.3 in the position 23467-23715 in cosmid MMDA (Access #M63796) which was automatically sequenced and contained PP2Cα first coding exon in position 59770 in MMDBC, GenBank accession #M89657, as well as 35-3.seg and 35-T7 as shown in FIG. 3. MMDA and MMDBC are two cosmids in the same contig. (More details on the sequences are found in Example 10 herein below).

Little is known about the role of PP2Cα in the cell or about its native substrates mainly because of the lack of specific inhibitors for PP2Cα. In Schizosaccharomyces pombe it has been shown that pp2cα-like enzymes are important for heat shock response and in osmoregulation [Shiozaki, 1995; Shiozaki, 1994]. In Saccharomyces cerevisiae pp2c-like activity has been implicated in the regulation of tRNA splicing and cell separation [Robinson et al., 1994]. In neural cells pp2cα might have a role in the regulation of the Ca++-independent activity of Ca++/calmodulin dependent protein kinase II (Fukugana, 1993]. Otherwise, information about pp2cα is scarce.

pp2cα might be a cell marker itself. The prior art does not provide information about pp2cα expression in tumor cells. There is a publication suggesting that pp2cα might have a role during myogenic differentiation. [Ohishi, 1992]. Based on the presence of a 10 amino acid motif which appears also in other transcription factors, pp2cα might function like a transcription factor and might regulate transcription in the cell under specific growth conditions and tissues. It can thus behave like E2F which is a major transcription factor and can act when improperly expressed either as an oncogene or as a tumor suppressor factor [Weinberg, 1996]

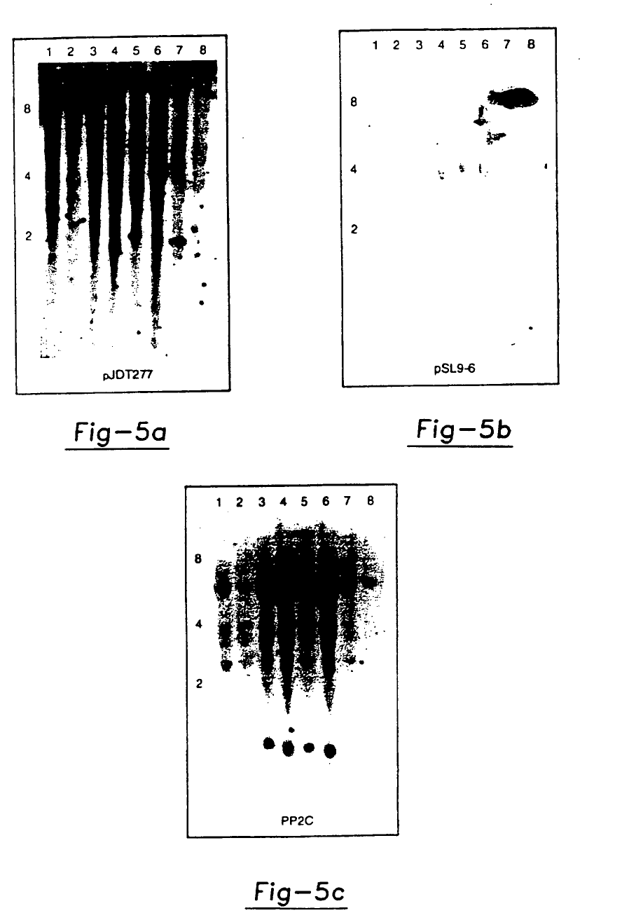

Unexpectedly, analysis of the PP2Cα mRNA in the AAV/neo cells demonstrated that the transcription of the gene was reduced upon treatment with DNA damaging agents in contrast to the parental SV40 transformed cells in which PP2Cα was induced following the treatment (FIG. 6).

The following is the densitometry analysis of the hybridization signals

| | CO60 | | CO60 T | | | C9-3T |
|---|---|---|---|---|---|---|
| | C | CO60 T | CO60 C | C9-3C | C9-3T | C9-3C |
| PP2Cα | 0.15 | 0.31 | 2.06 | 0.26 | 0.15 | 0.57 |
| rRNA | | | | | | |

The transcription of PP2Cα in C9-3 was reduced upon treatment with MNNG in contrast to the parental SV40 transformed cells in which PP2Cα was induced following treatment.

The intact cDNA coding for pp2cα was cloned from a cDNA library (provided by M. Oren, the Weizmann Institute of Science, Rehovot) and PP2Cα clone was stably introduced into the AAV/neo cells which lost the transformed phenotype following AAV-integration. The transformed features were rescued following the expression of the PP2Cα clone in the PP2Cα neo cells, the cells regained the properties of transformed cells, grew in soft agar and lost their apoptotic phenotype, but the cells could not be propagated. Similar cells which were transfected in the same efficiency with a control plasmid containing a truncated cDNA of non relevant gene did not regain the capability to grow in soft agar.

From these studies it is apparent that PP2Cα has a key role in the initiation and/or maintenance of transformed cells. It should be noted that though the cells grew in soft agar applicant was not able to isolate viable cells suggesting that unbalanced expression of the cells led to aberrant growth and to cell death.

PP2Cα appears to be important in development. The high conservation of the gene and protein throughout evolution, the specific control signals including IRES (internal ribosome entry site) at the 5′UTR support this. The findings by other laboratories that AAV infection effects specifically tumor cells might have two explanations: 1) The virus does not infect normal cells or cannot integrate into their genome in a specific manner. 2) Alternatively, if AAV integrates into PP2Cα in normal cells the disruption of this gene might not effect them or might be lethal not allowing the survival of such cells. The fact that the inactivation of only one allele of PP2Cα is responsible for the changes in the transformed phenotype and that the introduction of a functional PP2Cα cDNA clone rescues the transformed phenotype demonstrates the importance of PP2Cα. Applicant also noticed that in highly tumorigenic Chinese hamster cells derived from 9-3 that the whole chromosome carrying PP2Cα is duplicated 3, 4, and even 5 times.

In human, on the same chromosome, in close proximity to PP2Cα there is an important tumor specific marker called cancer embryonic antigen (CEA), which appears in most tumor cells. Both genes are mapped to chromosome 19q13.3. Targeting the treatment to cells carrying a marker like CEA should help. (It might be possible that CEA by itself is not relevant to cancer but it is associated with the enhanced expression of PP2Cα or duplication of this chromosomal region contains both genes).

The above discussion provides a factual basis for the use of PPC2 in cancer detection and therapy. The methods used with and the utility of the present invention can be shown by the following non-limiting examples and accompanying figures.

### EXAMPLES

### Methods

General Methods in Molecular Biology: Standard molecular biology techniques known in the art and not specifically described were generally followed as in Sambrook et al., Molecular Cloning: A Laboratory Manual, Cold Springs Harbor Laboratory, New York (1992), and in Ausubel et al., Current Protocols in Molecular Biology, John Wiley and Sons, Baltimore, Md. (1994). Polymerase chain reaction (PCR) was carried out generally as in PCR Protocols: A Guide To Methods And Applications, Academic Press, San Diego, Calif. (1990).

Reactions and manipulations involving other nucleic acid techniques, unless stated otherwise, were performed as generally described in Sambrook et al., 1989, Molecular Cloning: A Laboratory Manual, Cold Spring Harbor Laboratory Press, and methodology as set forth in U.S. Pat. Nos. 4,666,828; 4,683,202; 4,801,531; 5,192,659 and 5,272,057 and incorporated herein by reference.

Antibody Capture Assay: The steps of the method are: (1) binding of antigen to a solid phase; (2) binding of the antibody to the antigen; and (3) binding of a labeled secondary antibody to the complex. By binding constant amounts of rpp2cα to the solid phase, Applicants have used this technique to detect and quantitate monoclonal antibodies during the rounds of cloning, and to compare polyclonal antibodies from different rabbits and bleedings. The assay has also been used to detect pp2cαα in crude extracts of tissues and cell cultures.

Immunoblotting: The steps of the method are: (1) preparation of antigen sample; tissue extracts, cell culture extracts or rpp2cα preparations; (2) resolution of the sample by SDS-PAGE; (3) transfer of the separated proteins to a nitrocellulose membrane; (4) blocking nonspecific sites on the membrane; (5) incubation with poly- or monoclonal antibody; and (6) detection by labeled secondary antibody. Applicants have used immunoblotting for characterization of antibodies described herein above and for detection of pp2cαα in cell and tissue extracts. [Harlow and Lane]

Immunoprecipitation: The method steps are: (1) immobilization of monoclonal antibodies to a solid matrix (anti-mouse IgG conjugated agarose); (2) binding of antigen to immobilized antibodies; (3) resolution of bound proteins on SDS-PAGE; and (4) immunoblotting and detection of antigen by affinity purified rabbit polyclonal antibodies. The method has been used to estimate the amount and the molecular mass of different sized pp2cα and β polypeptides that were discovered.

pp2cα Activity Assay: PP2Cα gene product is purified from the mouse cells by general procedure, and its activity is assayed by its ability to dephosphorylate [32P] casein [McGowan and Cohen, 1988].

Oligonucleotide Primers for Reverse PCR: Rat PP2C-α cDNA specific primers were used for reverse transcription and PCR. These primers were obtained from General Biotechnology, Rehovot, and used without further purification. The primers' position is according to the rat kidney nucleotide sequence of PP2Cα cDNA reported by Tamura et al., [1989] in the Genbank (accession number: Gb_ro: Ratpp2c, J04503).

The approximate position of each primer on the rat PP2Cα cDNA:

| | |
|---|---|
| Primer #1: 5′ - AGGATCAAGTCATAATGGGA - 3′ (74-93nt - sense) | (SEQ ID No: 4) |
| Primer #4: 5′ - GCTGGAGTCTGATTTACAAC - 3′ (1454-1473nt - anti sense) | (SEQ ID No: 5) |

Antisense RNA: Artificial antisense RNA complementary to the PP2Cα gene is synthesized, and transfected to the mouse cells by the method of Inouye [1988].

Identification Of Unique Changes In Gene Expression By Differential Display: In order to detect changes in the expression of cellular genes mediated by AAV integration, the differential display method is used [Liang and Pardee, 1992; McClelliand et al., 1995]. This method is directed toward the identification of differentially expressed genes among approximately 15,000 individual mRNA species in a pair of mammalian cell population such as infected and uninfected cells, and recovering their cDNA and genomic clones. The strategy of the method consists of the following steps: (1) Reverse transcription in fractions using a set of anchored primers, (2) amplification of cDNA species from each fraction using a set of arbitrary primers and anchored primers by labeled PCR, (3) electrophoretic separation of the resulting fragments on sequencing gel, (4) reamplification of fragments that are different in the two situations, cloning and sequencing, and (5) confirmation of differential expression by an independent RNA analysis technique.

More specifically, total RNA is isolated from cells as described by Sambrook et al. The RNA is reverse transcribed with an oligo dT primer designed to bind to the 5′ boundary of the poly A tail. The cDNA is amplified in a PCR reaction with the oligo dT primer and a second 10-mer arbitrary in sequence. 40 cycles of PCR are done in the presence of [35S]-dATP, in the following conditions: 94° C. for 30 seconds, 42° C. for 60 seconds and 72° C. for 30 seconds. The amplified cDNAs are separated on a 6% sequencing gel, then exposed to X-ray film.

Bands of interest (bands that are differentially displayed) are cut out from the gel, and reamplified with the same primers as used to generate the original PCR product. To confirm differential regulation of individual candidate bands, Northern blot hybridizations is performed. Fragments of interest are cloned using a TA cloning Kit, and sequenced. Genes detected by this method are hybridized to Northern blots from the appropriate cells.

Chromatin Structure In Latently Infected Cells: Higher order chromatin structure may affect the transcription of cellular genes. Judging by their increased sensitivity to digestion with DNAseI or micrococcal nuclease, transcriptionally active chromatin regions are less tightly packed than chromatin containing transcriptionally inactive genes. Chromatin is partially purified and digested by micrococcal nuclease [Roth et al., 1990]. Purified DNA fragments are digested with a unique restriction enzyme to generate a series of fragments with one end defined by micrococcal nuclease and the other defined by the restriction enzyme. Fragments are separated by agarose gel electrophoresis, transferred to nitrocellulose filters and probed with labeled DNA fragments. Naked DNA is purified and processed similarly. Nucleosome position and nuclease sensitive regions are inferred by comparison of fragments from naked DNA and chromatin.

The methylation state of genes can indicate chromatin changes. Gene specific DNA methylation is measured by the methylation assay [Kafri et al., 1992]. In this method, total cellular DNA is digested with methyl-sensitive enzymes, such as HpaII or HhaI, and specific fragments of DNA that contain these sites are amplified by flanking oligonucleotide primers. If a specific site is methylated, the amplification will proceed normally. On the other hand, the presence of an unmethylated site will result n digestion of the fragment and the subsequent failure to visualize the amplification product. When properly calibrated, this assay is linear over a wide range of DNA concentrations and can be used to accurately measure the degree of DNA methylation at specific sites.

### Example 1

### Example of Studies with AAV

A. Infection of Mouse Cells with AAV:

A1. Generation of Mouse Cells with Stably Integrated AAV:

DA3 cells were infected with the JDT277 AAV/neo hybrid virus according to Winocour et. al. [1992], with slight modifications. Single colonies were isolated and amplified by serial passages in the presence of the antibiotic G418. The resistant cell lines were designated DA3J.

The DA3 cell line was derived from the in vivo D1-DMBA-3 mammary tumor syngeneic to BALB/c mice. The DA3 cell line produces tumors in BALB/c mice with the same growth kinetics and expresses the same tumor associated antigen (Ag) on its surface as the parental tumor. The cells express specific markers for tumor cells, and cease to express specific Ag typical to normal breast cells [Sotomayor et al., 1991].

To assess the influence of AAV on mouse cells, DA3 cells were infected with the JDT277 AAV/neo hybrid virus [Tratschin, 1985]. JDT277 contains the portion of the AAV2 genome, which encodes the Rep proteins, the AAV terminal inverted repeats (TIRs) and the prokaryotic neomycin phosphotransferase gene (neo), conferring resistance to G418. The neo gene was inserted at nucleotide 1882, resulting in carboxy terminus truncated Rep proteins. The truncation of the Rep proteins does not affect the ability of the AAV/neo virus to replicate in Adeonvirus coinfected human cells.

Single colonies were isolated and amplified by serial passages in the presence of G418. The resistant cells were designated DA35.

A2. Characterization of the AAV Genome in the DA3J Cells:

a. Southern Analysis

Genomic DNA isolated from DA3J1-DA3J7 clones was digested with different restriction enzymes (BglII or EcoRI), electrophoresed and hybridized to radiolabelled AAV DNA. The hybridization pattern is different in each clone, probably due to rearrangement of the AAV genome. Indeed it is known that integration of AAV DNA is frequently accompanied by alterations within the viral sequences [Walz and Schlehofer, 1992].

b. PCR Analysis

To find whether the rep promoters and ORF's were present in the DA3J cell lines, PCR reactions were carried on 13 clones (DA3J1-DA3J13), using different oligonucleotide primers complementary to the AAV and neo sequences. The results demonstrated that parts of the viral sequences in each clone were somehow interrupted. In all the examined cell lines the AAV rep ORF's were not intact, thus, impairing the expression of the AAV specific proteins.

In two clones, DA3J3 and DA3J4, two AAV molecules integrated into the host genome in a head-to-tail pattern. This finding is in agreement with earlier studies showing that the AAV DNA recombined into the Chinese hamster host DNA, at least in some cases, as a head-to-tail concatamer of more then one viral genome, via the terminal sequences of the viral molecule [Cheung et al., 1980; Walz and Schlehofer, 1992]. The integrated AAV from DA3J7 served as a silencer for SV40 replication in 293 cells in a similar assay to that described in Example 7 herein below.

A3. Expression of AAV Genes in the Infected DA3J cells:

Cell extracts from the infected DA3 were prepared according to Winocour et al. [1992]. Samples from the extracts were electrophoresed on a 12% PAGE, and immunoblotted with an anti-Rep antibody. The results showed that, the two major Rep proteins, Rep78 and Rep68, are not expressed in any of the infected cells, however one of the small Rep proteins, Rep40, is expressed in two clones, DA3J11 and DA3J13. These results were expected from the PCR analysis that showed that in all the examined cell lines the rep ORF's were not intact.

B. Site Specific Integration

B1. Comparison of the Integration Site of AAV in the Mouse Cells with the AAV Integration Site in the Chinese Hamster Cells:

Genomic DNA from parental DA3 and DA3J clones (DA3J1-DA3J7) was digested with BglII or EcoRI. Following electrophoresis the blots were hybridized once with the cellular sequence from the virus/cell junction, isolated from C9-3 (psL9-6), and once with radiolabelled AAV DNA. In three of the cell lines (DA3J3, DA3J4 and DA3J6) the cellular probe and the AAV probe hybridized to common bands. Using PP2Cα probe applicant found in BglII digested DNA that both AAV and PP2Cα hybridized to the same bands. Thus, both in Chinese hamster and mouse, AAV always integrated into the same site in the vicinity of PP2Cα. Note that in all cell lines including the parental DA3 cells, PP2Cα and the cellular clone 6 probes hybridized to the same band which is the preintegration site.

C. Effect of AAV on the Cellular Phenotype:

The following cytological properties were compared between DA3 infected and parental cells:

a. Plating Efficiency

As shown in Table 1, the plating efficiency of the DA3J cells was reduced compared to the plating efficiency of the parental DA3 cells, by 11% (DA3J2) to 54% (DA3J3).

b. Sensitivity to UV Irradiation

As shown in Table 2, the DA3J cells show increased sensitivity to UV irradiation compared to the parental DA3 cells. There is a decrease of 5i to 55% in the survival rate of the DA3J cells compared to the DA3 cells.

These results are in agreement with other studies, which demonstrated that AAV infected cells (Hela, CO60) show reduced plating efficiency, and enhanced sensitivity to UV irradiation, compared to uninfected cells [Walz and Schlehofer, 1992; Winocour et al., 1992].

It is interesting to note that, DA3J3 shows the lowest plating efficiency, and the highest sensitivity to UV irradiation. This may be due to the fact that DA3J3 contains two integrated AAV molecules, while DA3J1 and DA3J2, contains only one.

c. FACS Analysis

FACS analysis was performed on DA3, DA3J1, DA3J2, and DA3J3 as described by Vindelov et. al., [Vindelov et al., 1983]. In this procedure no significant changes were observed between the parental DA3 cells and the DA3J cells.

### Example 2

### Cloning and Expression of PP2Cα

Full length of the coding region of PP2Cα cDNA was isolated from a rat cDNA library. The cDNA was cloned into the expression vector pET-17b (pET System, Novagen) between the Kpn1 and Not1 restriction sites. E. coli cells (BL21-DE3) transformed by the expression plasmid yielded high levels of recombinant pp2cα (rpp2cα) as observed by a very prominent ˜45 kDa band on SDS-PAGE.

Assay of pp2cα activity: Protein phosphatase activity was found in crude extracts of cultures harboring the recombinant plasmid, as measured by the method of McGowan and Cohen [Methods Enzymol. 159: 416-429, 1988].

Purification of rip2cα: Overnight cultures were grown at 30° C. in LB medium containing ampicillin. The cells were harvested and disrupted by sonication. The sonicate, cleared by centrifugation, was further purified by ammonium-sulfate precipitation and anion exchange chromatography on DEAE-sephadex.

### Example 3

### Production and Analysis of Antibody

Polyclonal Antibody preparation in rabbits: Crude cell extracts containing ˜250-500 μg rpp2cα were separated on 12% preparative SDS-PAGE (200×150×1.5 mm). The rpp2cα band, located by side-strip staining, was excised and stored at −20° C. For injection in rabbits, the band was fragmented by repeated passage through a 18 gauge syringe needle and mixed with an equal volume of Freund's adjuvant. Four ml of emulsion originating from one SDS-PAGE band were used to inject two rabbits. The rabbits, which were pre-bled, were injected subcutaneously at four week intervals. For primary immunization complete Freund's adjuvant was used and all other injections were in incomplete Freund's. The animals were bled every two weeks and the serum was cleared by centrifugation. The antibodies were designated 351 and 343. Unless otherwise designated work described herein used 351.

Monoclonal antibody preparation: Purified rpp2cα was used for monoclonal antibody preparation, by mouse hybridoma production as described herein above. Hybridoma colonies were screened by antibody capture assay (see herein below) and by immunofluorescent cell staining. Positive colonies were subjected to two rounds of cloning and screening by the same methods. Finally, eight (8) positive clones were chosen for further study. Antibodies from these clones were collected as tissue culture supernatants and also as ascitic fluid.

Antibodies Developed Against pp2cα and pp2cβ

(1) A rabbit polyclonal designated 801 raised against the carboxy terminal peptide (10 a.a.). This antibody recognizes pp2cα and not pp2cβ.

The antibody was raised in rabbits and was affinity purified against the rpp2cα. This antibody was used in most of the histochemical analyses.

A rabbit polyclonal antibody raised against PNKDNDGGA (SEQ ID No:3), the carboxy terminal of pp2cβ.

(2) A rabbit polyclonal designated 351 raised against rpp2cα which recognizes epitopes on both α and β.

(3) Eight independent monoclonal antibodies which were raised against the rpp2cα and were screened and chosen according to their reaction in ELISA with the rpp2cα and by their capability to recognize pp2c (α and β or α) by immunofluorescence staining of hepatoma cells by Western blotting and immunoprecipitation.

Table 4 provides the characterization of eight monoclonal antibodies by antibody capture assay, immunoblotting and immunoprecipitation. The combination of these assays allow the isolation of monoclonal antibodies with the proper specificity of the present invention.

### Example 4

Expression of pp2cα in normal Breast tissue and Breast tumors: Paraffin blocks obtained from normal breast and breast carcinoma were stained with 801 antibodies specific to pp2cα and then with secondary antibodies coupled to peroxidase. The substrate was DAB. The samples were counterstained with methylene blue. In a few experiments, the antibodies used were monoclonal 2A3 which are specific to pp2cα. The magnification was ×400. Normal liver and hepatoma tissue and normal colon and colon carcinoma tissue was also tested.

In the normal and hyperplastic breast samples, the nuclei were stained very predominantly with the antibodies. In breast carcinoma, there was a predominant staining in the cytoplasm. In invasive carcinoma, no staining with anti-pp2cα was observed. Interestingly, in liver tissue cultured cells, when stained with 801 or 2A3 antibodies, the cell surface was stained indicating that PP2Cα gene product was expressing in the cell membrane. In normal liver cells a very pronounced cytoplasmic staining with few very strong nuclear regions stained. In the hepatoma fainter cytoplasmic staining and less staining in the nucleus was observed.

It should be noted that it appears there is a differential loss of pp2cα as the cells are advanced in their malignant appearance in the counterstaining.

### Example 5

Expression of pp2cα in normal Colorectal tissue and Colorectal tumors: The level of the protein phosphatase 2Cα (pp2cα) expression was assessed in colorectal cancer tissues in comparison to normal colon tissues and in Chinese hamster embryo (CHE) cell line in comparison to the non permissive SV40 transformed Chinese hamster cells (CO60), by the reverse transcriptase polymerase chain reaction assay (RT-PCR).

Samples of 1 μg total RNA were denatured at 65° C. for 10 minutes in the presence of 0.5M oligo dT (15 mer) as an anti-sense primer, and immediately chilled on ice. First strand cDNAs were obtained after 60 minutes at 37° C. in a 50 μl reaction mixture containing: 0.25 mM dNTPs (Promega), 10 mM DTT, 20 u RNasin, 50 u MMLV reverse transcriptase and 5 μl of 10×reaction buffer (STRATAGENE). Following inactivation at 95° C. for 10 minutes, 3 μl of the resulting cDNA were used in a 100 μl PCR reaction containing: 0.025 mM dNTPs (PROMEGA), 10 mM DTT, 20 U RNasin, 50 U MMLV reverse transcriptase and 5 μl of 10×reaction buffer (STRATAGENE).

Following inactivation at 95° C. for 10 minutes, 3 μl of the resulting cDNA were used in a 100 μl PCR reaction containing: 0.025 mM dNTPs (PROMEGA), 0.5 μM of PP2Cα sense primer 5′-GAAGTAGTCGACACCTGT-3′ (SEQ ID No:6), 0.5 μM of PP2Cα anti-sense primer 5′-GCTGGAGTCTGATTTACAAC-3′ (SEQ ID No:5), 10×reaction buffer, 2.5 mM MgCl₂ and 2.5 u of ABTaq polymerase (Advanced BioTechnology). Thirty-five cycles of PCR were performed in the following conditions: 1 minute at 94° C., 1 minute at 60° C., 1 minute at 72° C., followed by 72° C. for 10 minutes. The same cDNAs were used as templates for parallel PCR reactions performed in the presence of β-actin primers 5′GTTTGAGACCTTCAACACCCC-3′ (SEQ ID No:7) and 5′GTGGCCATCTCTTGCTCGAAGTC-3′ (SEQ ID No:8), in the same PCR reaction mixtures. Aliquots were taken after 20, 25, 30 and 35 cycles and analyzed by gel electrophoresis.

Results

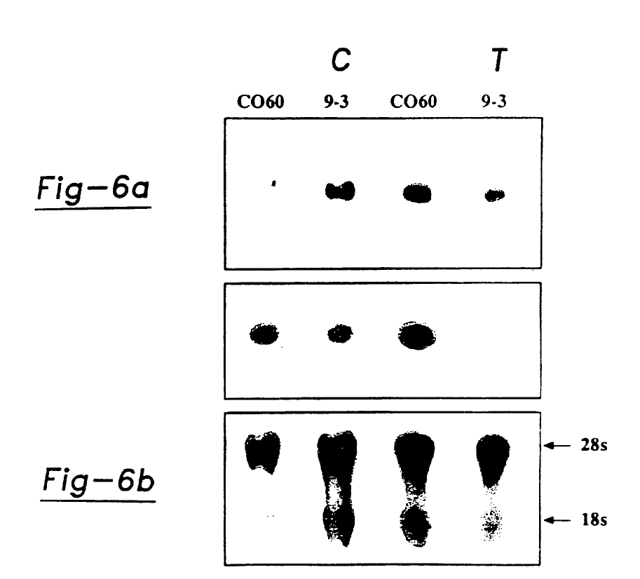

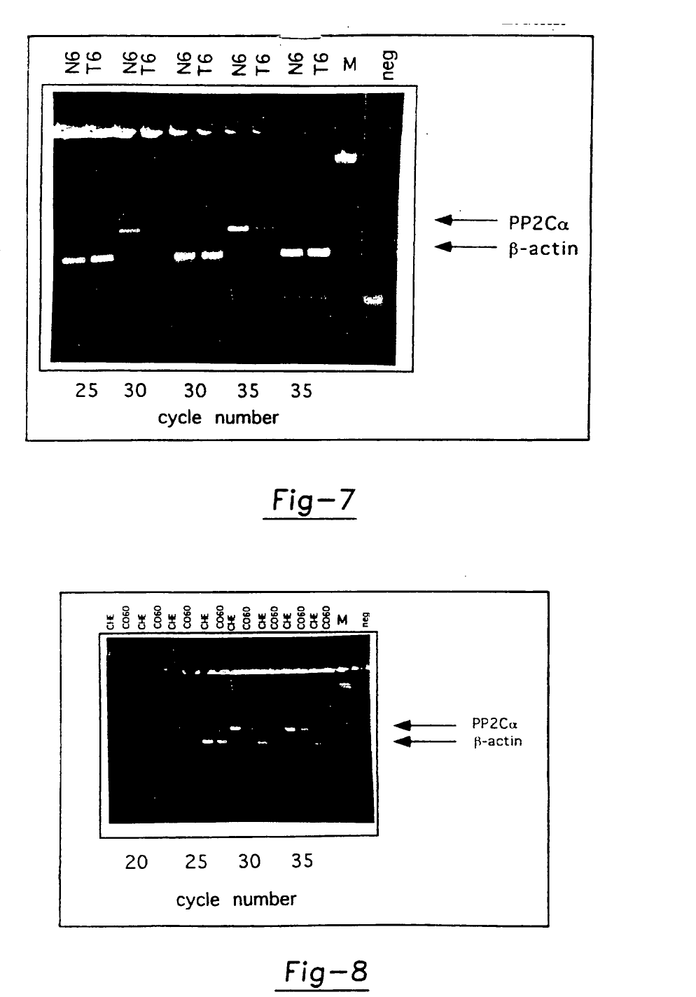

The use of PP2Cα specific oligonucleotide primers, generated RT-PCR products with the expected size of 480 bp. The RT-PCR reactions demonstrated that in 7 out of 8 samples, the level of PP2Cα mRNA were significantly higher in normal colon tissues than the levels obtained in the adjacent tumor colonic tissues. (FIG. 7). The level of PP2Cα expression in CHE cell line was higher than in the CO60 cell line (FIG. 8).

### Example 6

### Production of Vectors and Transformed Cells Harboring the Vector

Expression of the PP2Cα mRNA under the inducible tet promotor: Expression of the sense and antisense PP2Cα mRNAs in mammalian cells is based on the system for tetracycline-regulated inducible gene expression as described by Gossen and Bujard [1992].

This system relies on constitutive expression of a tetracycline-controlled transactivator (tTA) fusion protein which combines the tetracycline repressor with the activating domain of herpes simplex VP16. The tTA was constitutively expressed in rat fibroblasts and in HeLa cells. In these two cell lines the tTA stimulates transcription from a minimal promoter derived from the human cytomegaloviruspromoter and the tetracycline operator. Upon addition of tetracycline the stimulation of transcription by tTA is inhibited.

Clones were prepared by stable transfection of the two cell lines with expression vectors that contain the PP2Cα mRNA in the sense and in the antisense orientation, under the control of the tTA-dependent promoter.

Construction of expression vectors: The construction of the plasmids used as expression vectors included the following steps.

1. Preparation of the DNA fragment coding for the rat PP2Cα mRNA.

2. Cloning of the rat PP2Cα cDNA into the tTA containing plasmid.

3. Verification of the plasmids by restriction map analysis.

4. DNA sequence analysis of the PP2Cα cDNA insert.

Preparation of the DNA fragment coding for the rat PP2Cα mRNA: The DNA fragment coding for the rat PP2Cα mRNA was prepared by thermal cycling amplification. The template for the amplification reaction was the insert of plasmid skPP2C (PP2Cα cDNA cloned into the sk BLUESCRIPT plasmid).

The upper primer used in the amplification reaction contains the sequence coding for the first six amino acids of the rat PP2Cα (Met Gly Ala Phe Leu Asp; SEQ ID No:9). The sequence of the upper primer is the following: 5′ CGGGATCCGC ATGGGAGCAT TTTTAGAC 3′ (SEQ ID No:10).

The lower primer used in the amplification reaction contains the sequence coding for the last five amino acids and the stop codon of the rat PP2Cα (Thr Asp Asp Met Trp ***; SEQ ID No:11). The sequence of the lower primer is the following: 5′ CGCGGATCCT TACCACATAT CATCAGT 3′ (SEQ ID No:12).

The ends of the DNA fragments were modified by introduction of BamHI restriction sites at both ends. Cloning of the rat PP2Cα cDNA into the tTA containing plasmid: Following amplification of the rat PP2Cα cDNA, the DNA fragment was cleaved with restriction enzyme BamHI and cloned into plasmid pUHD10-3 [Gossen and Bujard, 1992] downstream from the tetracycline responsive promoter.

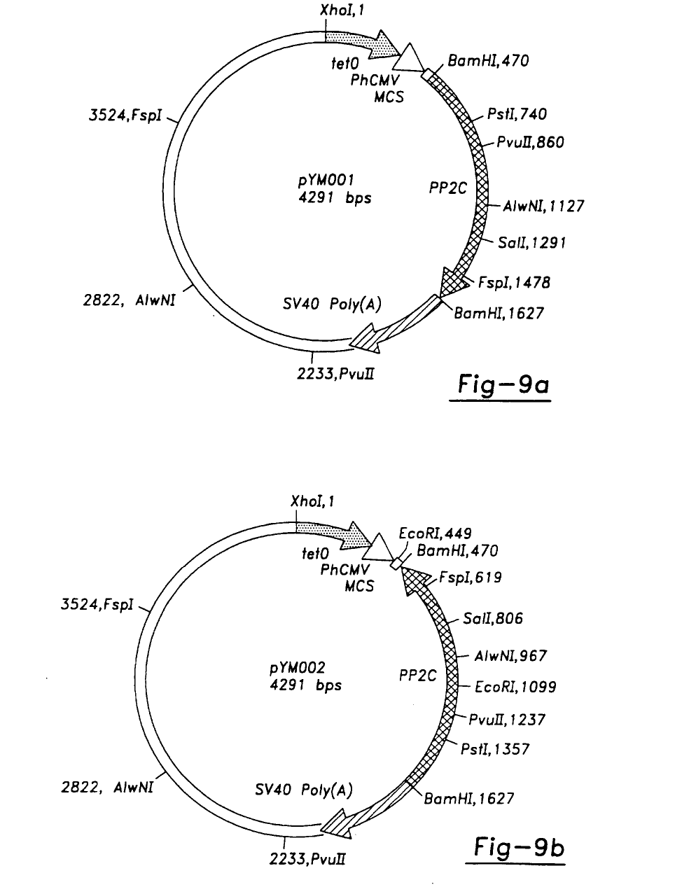

Verification of the plasmids by restriction map analysis: The orientation of the cDNA insert with respect to the promoter was determined by restriction map analysis. Plasmids that contain the cDNA in the sense orientation (pYM001) and in the antisense orientation (pYM002) were selected (FIG. 9).

DNA sequence analysis of the PP2Cα cDNA insert: The sequence of the DNA insert of plasmid pYM001 was determined by automatic DNA sequence analysis. The primers used for sequencing analysis were the same as the one used for cloning. The results of this analysis show that the sequence of the cloned fragment is identical to that of rat PP2Cα and that no mutation was introduced during the amplification reaction.

Transfection: Plasmids pYM001 and pYM002 were introduced in the rat fibroblast and in the HeLa cell lines which constitutively express the tTA, by CaPO₄ coprecipitation with plasmid pBSpac. Plasmid pBSpac contains a genetic selective marker, that confers puromycin resistance.

Twenty-four to forty-eight hours following transfection, cells were passaged 1:10 and grown in selective medium. The selective medium for HeLa and for the rat fibroblast cell lines contained 0.3 (μg/ml and 1 μg/ml, neuromycin respectively. After two to three weeks clones were isolated, grown to confluence in 24 wells culture plates, transferred to 10 cm dish, grown to 70-90% confluence and frozen in 90% fetal calf serum 10% DMSO. In these clones following the removal of Tet we observed induction in PP2Cα mRNA in clones harboring pYM001 and reduction in the endogenous mRNA of PP2Cα in a clone harboring the antisense plasmid pYM002.

### Example 7

### Role of Integrated AAV in the Modulation of PP2Cα Expression

This work does not identify the precise site of AAV integration within the gene. Additionally, the data does not provide the exact site for the human integration site which is also located on the same chromosomal region 19q13.3 but not within the 106 kb cosmid. Applicants hypothesize that based on the results with RNA and the specific protein that the AAV integration site is either in the gene coding for pp2cα or its regulatory region is in some way associated with PP2Cα. For example, rep might interact with pp2cα protein and the AAV genome linked to rep and PP2Cα might be associated with the regulatory region of PP2Cα.

In stable cell lines, SV40 amplification was suppressed by infection with recombinant AAV/neo virus and Applicants were not able to detect the expression of rep or the sequences coding for rep and, therefore, Applicants searched for a sequence with suppressing activity. This sequence was present in all the AAV harboring cells whether they are of Chinese hamster or mouse origin.

A functional correlation between the integrated AAV sequences and the modulation of PP2Cα activity in the cells harboring the AAV genome was not demonstrated yet, however, it is clear that as a result of the AAV integration, there was an alteration in the transformed characteristics as described herein.

Based on the studies described below, it seems feasible that in addition to the site specific integration which occurs in the vicinity of PP2Cα, the AAV sequences can have some importance in the alteration of the transformed phenotype.

The integrated AAV in SV40 transformed Chinese hamster cells (line 9-3 and other cell lines) is responsible for the suppression of the carcinogen induced Sv40 amplification. The viral element responsible for the suppression of SV40 amplification (silencer, SEQ ID No:13) was defined using a transient assay for SV40 replication [Yang et al, 19951] to demonstrate that the AAV rep protein is responsible for the suppression of SV40 replication. This assay is based on transfection of the human kidney cell line 293 with an SV40 vector containing the coding region for T antigen and the viral origin of replication and cotransection with several different constructs derived from the vicinity of AAV integration in 9-3, SV40 transformed Chinese hamster embryo cells containing an integrated AAV and DA357 (a mouse cell line harboring the integrated AAV in which the transformed phenotype was altered following AAV integration). Using different constructs, Applicants succeeded to define the minimal AAV element conferring the suppression. This element is comprised of 64 nucleotides from the AAV genome (nucleotides 125-189).

ACTCCATCACTAGGGGTTC [symbol] TGGAGGGGTG
GAGTCGTGACGTGAATTACGTCATAGGGTTAGGG

This element was termed SV40 silencer (SEQ ID No:13) though in an alternative embodiment only 21 nucleotides, 125-145, (SEQ ID No:14) are responsible.

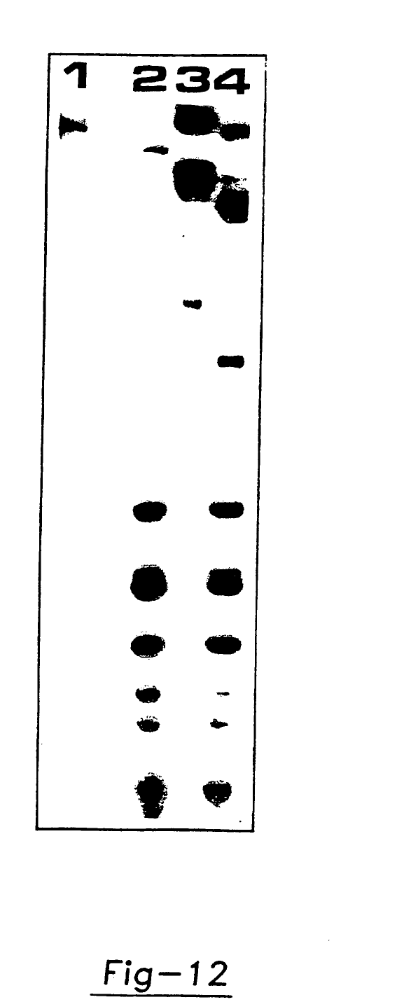

The replication of the SV40 vector in 293 cells results in DNA which is not methylated and, therefore, is cleaved by DpnII, an enzyme that cleaves only unmethylated DNA. The DpnII digested DNA was separated on a gel, blotted and hybridized with SV40 probe. The results are shown in FIG. 12.

The blot provides the following:

Lane 1: Cotransfection with pSK1 and pAV2 (a plasmid containing the whole AAV genome and expressing the rep protein). Note that SV40 replication is suppressed.

Lane 2: Transfection with the SV40 plasmid pSK1 SV40 replicates. Replication of the SV40 template is observed.

Lane 3: Cotransfection of pSVK1 and a plasmid harboring 138 bp from the AAV genome (nucleotides 125-263). There was a suppression of SV40 replication by this element.

Lane 4: Cotransfection of pSVK1 with pSL9-6 (non AAV DNA sequences).

Thus SV40 replication in 293 cells was suppressed by rep and by the silencer element.

Similarly SV40 replication was suppressed when the cells were transfected by a plasmid containing only 125-145 (the SV40 mini-silencer, SEQ ID No:14).

5′ A₁₂₄ C.₁₂₅TCCCATCACTAGGGGTTCCT,₄₅.

In control experiments in which Applicants transfected with other sequences derived from the integrated AAV and cellular sequences spanning the integrated AAV, such as pSL9-6 and others (see FIG. 12, lane 4) no repression of SV40 DNA replication was detected.

Using a λ clone containing the integrated AAV and the flanking cellular sequences from the mouse DA3J7 cell line similar suppression of SV40 replication was observed. This clone contains the 64 nucleotides comprising the silencer region.

Note that suppression of SV40 replication can be obtained in 293 cells by Rep expression and by the 64 bp silencer element in a transient assay.

Revertants which have lost the integrated AAV regained the capability to amplify SV40. In one revertant line, C9-3-2, applicant showed that the revertants lost the whole chromosome containing the integrated AAV. Applicants showed this by FISH, the disappearance of a very well characteristic abnormal chromosome into which AAV integrated.

A hypothesis for the above observations can be made, but it is not to be construed as limiting the present invention to this one mode of action. Applicants propose that the Rep protein [Heilbronn, Schlehofer et al, 1983; Kleinschmidt et al, 1995; Yang et al, 1995] and the silencer element can suppress SV40 replication by interaction with a similar protein or element directly or indirectly, possibly PP2Cα

It is possible that the 21 bp (mini-silencer) from AAV genome modulates PP2Cα activity as well by interaction and activation of a control region. Alternatively, the silencer can act as a dominant negative element interacting directly and/or indirectly with proteins associated with the replication of SV40. PP2C can regulate the action of such proteins by dephosphorylation. An example for such interplay can be the DNA polymerase a primase. It is possible that the rep protein is directly also involved in such interactions.

It appears that dephosphorylate DNA polymerase α primase is responsible for the initiation of SV40 DNA replication during the carcinogen induced amplification in CO60 cells. Moreover that this phosphorylate—dephosphorylate process are controlled by the cell cycle. Thus PP2Cα can modulate the activity of the DNA polymerase a primase depletion of PP2Cα due to its binding to rep directly or indirectly might lead to aberrant phosphorylic of the DNA polymerase a primase and to its failure to initiate SV40 DNA replication.

### Example 8

### There are more pp2cα Proteins than the 42 kd

Liver extracts were immunoprecipitated with the different monoclonal antibodies raised against rpp2cα (see Example 3 herein above). The precipitates were divided into two aliquots which were separated on 12% PAGE and blotted. Each set of immunoprecipitates was challenged with the following polyclonal antibodies:

(1) 351—which recognizes both α and β

(2) 801—specific to pp2cα

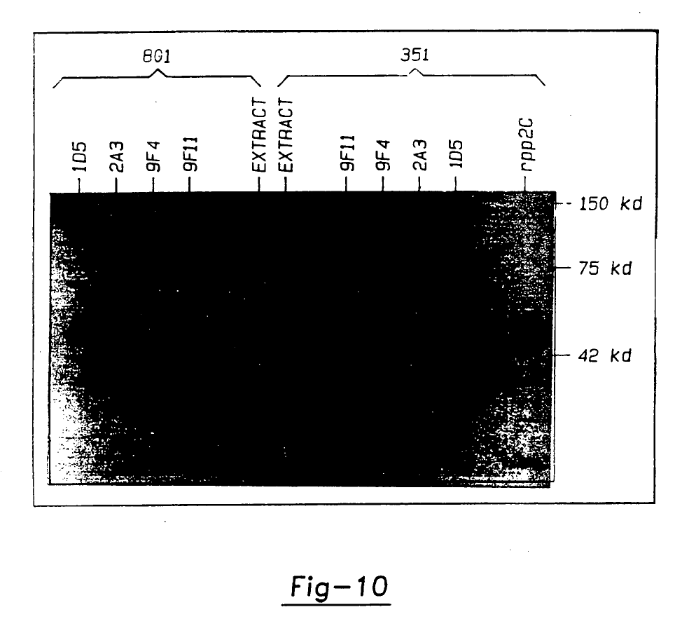

As demonstrated in FIG. 10 upon reaction with antibody 351 several bands migrating in the position of 40-43 kd were detected. Monoclonal antibodies 9F11, 9F4 and 1D5 displayed a very similar picture of 2-3 strong bands and 1-2 faint bands. Monoclonal antibody 2A3 precipitated only the faint bands.

The second part of the blot was reacted with the α specific antibody 801. In the position of ˜40-43 kd, two bands were visible with all four monoclonal antibodies. These bands are probably those which were detected by monoclonal 2A3. Thus, monoclonal antibody 2A3 is specific for pp2cα.

In addition, two additional bands migrating in the position of 75 kb and greater than 150 kd were detected. These proteins are more abundant in liver than the 40-43 kd protein. Since all eight monoclonal antibodies recognized these proteins and since the 801 polyclonal antibody reacted as well, it is clear that these two large proteins share several epitopes with pp2cα suggesting that they are the product of the same gene but result from alternative splicing.

A weak reaction with the 75 kd protein is detected with polyclonal antibody 351 when reacted with the immunoprecipitate. However, upon direct immunoblotting of total liver extract, it seems that 75 kd was present and a faint reaction could be detected against the higher molecular weight protein.

Several additional higher molecular weight proteins were also detected with polyclonal antibody 351. These proteins did not interact with polyclonal antibody 801, which is specific to pp2cα. These results suggest that other forms of α exist and have different molecular weights.

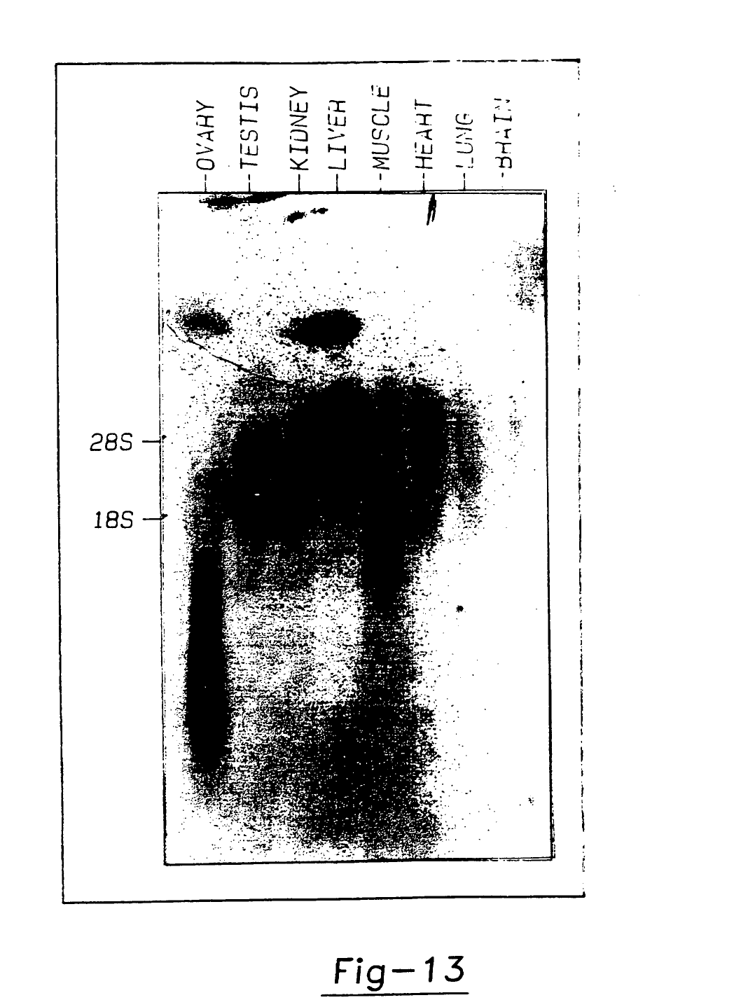

Norther blot analysis (FIG. 13) of mRNA derived from several tissues displayed several bands of RNA which hybridized with probes derived from the 5′UTR of pp2cα or with the entire PP2Cα cDNA probe. The RNA was extracted from different tissues and different sizes of RNA appeared in these tissues.

### Example 9

### pp2cα Regulates mRNA Synthesis

Table 6 summarizes the protein or RNA sequence homology that was found for the 10 amino acid pp2cα carboxy terminal peptide: NDDTDSASTD (SEQ ID No:1). This peptide was used to raise polyclonal antibody 801 as described herein above.

The carboxy cellular domain (CTD) of the RNA polymerase II fused to GST and bound the fusion complex to sepharose gluthation beads and mixed with HeLa cell extract. Following PAGE and blot the proteins bound to the carboxy terminal domain (CTD) of the RNA polymerase bound also to pp2cα. Both 801 and 2A3 were used with the blot. The size of the associated pp2cα was approximately 43 kD. Thus, RNA polymerase II is associated with pp2cα.

In a second experiment, extracts from tumor cells, Hepatoma and HeLA, were assayed as above. Binding was observed to GST-CTD only in the tumor extracts while in cell extracts from normal heptocytes such activity was not detected.

Based on the studies [Chambers and Dahmus, 1994] which demonstrated that the polymerase α CTD domain can be dephosphorylated by a phosphatase similar in its properties to PP2Cα (but not PP2Cα) applicant proposes that pp2cα dephosphorylates RNA polymerase II and thus regulates the initiation of mRNA synthesis on specific messenger. This peptide can be used to control and regulate transcription facilitated by other factors.

### Example 10

### Further Sequences

Two λ clones containing the AAV integration site were prepared. (1) One was derived from the chinese hamster cell CO60 designated λSL9-1 (schematic diagram in FIG. 3; SEQ ID Nos:15 and 16). Parts were sequences as indicated in FIG. 3. A further sequence AN8T7 (SEQ ID No:18) was derived from plasmid pSL9-8 (FIG. 3). (2) The second λ phage was cloned from the cell line DA3J7 and was not mapped. Portions were subcloned to plasmids and part sequenced as set forth in 5h-1 (SEQ ID No:17) A sequence comparison shows that 5h-1 is homologous to AN8T7. This region of comparison was also homologous to the cosmid MMDA 23,467-23715. Throughout this application, various publications are referenced by citation and patents by patent number. Full citations for the publications are listed below. The disclosures of these publications in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

TABLE 1
Plating efficiency of the DA₃J clones compared
with that of the parental DA₃ cell line

| No. of | No. of outgrowing colonies (mean ± | | | |
|---|---|---|---|---|
| cells | SD), % of plating efficiency | | | |
| plated | DA₃ | DA₃J₁ | DA₃J₂ | DA₃J₃ |
| 250 | 88 (6) | 53 (8) | 78 (11) | 43 (5) |
| | 35% | 21% | 31% | 17% |
| 500 | 181 (36) | 103 (10) | 131 (20) | 82 (9) |
| | 36% | 20% | 26% | 17% |

Semiconfluent cells derived from DA₃, DA₃J₁, DA₃J₂, and DA₃J₃ cell cultures were seeded at 250 and 500 cells onto 9-cm plastic petri dishes. Cultures were grown for 7 days. Cells were than fixed and stained with Gimsa. The mean number of growing colonies from two experiments was determined. Each experiment was performed in triplicates.

TABLE 2
UV sensitivity of the DA₃J clones compared
with that of the parental DA₃ cell line

| | No. of outgrowing colonies (mean ± SD), % of survival | | | | | | | |
|---|---|---|---|---|---|---|---|---|
| J/m² | DA₃ | | DA₃J₁ | | DA₃J₂ | | DA₃J₃ | |
| 0 | 91 (4) | 100% | 59 (20) | 100% | 79 (6) | 100% | 36 (7) | 100% |
| 2.5 | 86 (9) | 95% | 54 (9) | 91% | 85 (8) | 100% | 36 (4) | 100% |
| 5 | 62 (5) | 68% | 25 (30) | 42% | 39 (13) | 49% | 11 (11) | 30% |
| 10 | 22 (6) | 24% | 12 (2) | 20% | 19 (4) | 24% | 4 (2) | 11% |
| 20 | 0 (0) | 0% | 0 (0) | 0% | 2 (2) | 2% | 0 (0) | 0% |

Semiconfluently growing cells from DA₃, DA₃J₁, DA₃J₂, and DA₃J₃ cell cultures were seeded at 250 cells onto 9-cm plastic petri dishes. After 48 hours the cultures were irradiated with UV light, and then incubated for 7 days. The mean number of growing colonies was determined. The experiment was performed in triplicates.

TABLE 3A

| | |
|---|---|
| Fite: Shu . . . 008 | Acquisition Date: 10-May-95 |
| Total Events: 7000 | X Parameter: FL2-A (Linear) |

| Marker | Events | % Total | Mean | CV |
|---|---|---|---|---|
| All | 7000 | 100.00 | 384.11 | 48.19 |
| G0/G1 | 3355 | 47.93 | 394.88 | 14.49 |
| S | 943 | 13.47 | 569.28 | 7.41 |
| G2 + M | 668 | 9.54 | 727.76 | 8.06 |
| Ap. | 1485 | 21.21 | 132.36 | 36.21 |

TABLE 3B

| | |
|---|---|
| File: Shu . . . 010 | Acquisition Date: 10-May-95 |
| Total Events: 7000X | Parameter: FL2-A (Linear) |

| Marker | Events | % Total | Mean | CV |
|---|---|---|---|---|
| All | 7000 | 100.00 | 382.46 | 51.58 |
| G0/G1 | 3019 | 43.13 | 393.54 | 14.79 |
| S | 938 | 13.40 | 574.35 | 7.10 |
| G2 + M | 798 | 11.40 | 737.43 | 8.24 |
| Ap. | 1684 | 24.06 | 133.69 | 36.68 |

TABLE 4
Characterization of Monoclonal Antibodies

| | Antibody | | | |
|---|---|---|---|---|
| Antibody | capture⁽¹⁾ | | | |
| number | PP2C | PET | Immunoblotting⁽²⁾ | Immunoprecipitation⁽³⁾ |
| 1D5 | +1/8 | − | +++ | +++ |
| 2A3 | +1/32 | − | +++ | α-specific |
| 2H8 | +1/16 | +1/8 | not tested | +++ |
| 9F4 | +1/10 | − | +++ | +++ |
| 9F11/169 | − | − | nonspecific | +++ |
| 9F11/53 | +1/10 | − | +++ | +++ |
| 10C6 | +1/10 | +1/10 | ++ | +++ |
| 10F8 | +1/1 | − | + | +++ |

⁽¹⁾Antibody capture assay was performed on two antigens: purified rpp2c - to identify anti pp2c antibodies, and a protein extract from cells containing the pET expression vector without the pp2c insert - to identify non-specific antibodies. Results are expressed as + or − and the number indicates the antibody dilution giving the highest result.
⁽²⁾Immunoblotting tested on rpp2c and on liver extracts separated on 12% SDS-PAGE.
⁽³⁾Immunoprecipitation. Monoclonal antibodies were used to precipitate proteins from a liver extract. The proteins were separated on 12% SDS-PAGE and detected by affinity purified rabbit polyclonal antibodies - 801 which is α-specific and 351 which identifies pp2cα and pp2cβ.

TABLE 5

Homology between CHINT (SEQ ID No: 19) and
a sequence from human DNA from cosmid DNA
MMDB (SEQ ID No: 20) containing the 5′ end
of the PP2C RNA

| | |
|---|---|
| LOCUS | HUMMMDBC 68505 bp ds-DNA PRI 09-APR-1992 |
| DEFINITION | Human DNA from cosmid DNA MMDB (f10080) and MMDC (f13544) from |
| | chromosome 19q13.3 (obtained by automated sequence analysis). |
| ACCESSION | M89651 M77823 M77824 M77825 |
| KEYWORDS | |
| SOURCE | Homo sapiens (library: Lawrist5 vector library of A.V. Carrano) |
| SCORES | Initl: 61 Initn: 150 Opt: 82 |
| | 58.3% identity in 103 bp overlap |
| | 20 30 40 50 60 70 |
| int.li | TAGTGCCGGTCAAGGAACTGAACGTGCGATTCCGGGACAGGCTACCCACTCCGATCCCAG |
| | \|\| \| \| \| \|\|\|\|\|\| \|\|\|\|\|\|\|\|\|\| \|\| \|\|\| |
| hummdb | CCTCACCTCCGCCCTGTTTCGTCCAGGTCCTCCGGGTCAGGCTACCCCCGTCGCCGCCA- |
| | 57710 57720 57730 57740 57750 57760 |
| | 80 90 100 110 120 130 |
| int.li | GAGAAGTTGTCATGGTGAGGGCCACCCTAGGTCTCTGCCCCTGCTGTGTCCCCCATCTTA |
| | \|\|\| \| \| \|\| \|\|\| \|\| \|\| \| \| \|\| \|\|\| \|\| \|\|\|\|\|\|\|\| |
| hummdb | GAG-CGCGGGGGAGGGGAGAGCTTCCTTTGTCTCCTATGCCTCCT---CCCCCCATCCCG |
| | 57770 57780 57790 57800 57810 57820 |
| | 140 150 160 170 180 190 |
| int.li | CCCATCCAGTAGGATCTAGAGGCTGTCGCCCCCTTGTGGAATGCACAGAAGTCACAAGCG |
| \| \|\|\| \| \|\| \| \| \| \| \|\| \| \| \| \| \| \| \| | |
| hummdb | GCTCTCCTGCGGGCAAGCGCCGAGGGGACACCGGGGAGTACCCCACCTGAACCTCTGGGG |
| | 57830 57840 57850 57860 57870 57880 |

TABLE 6
Proteins or RNA Sequences Homologous to NDDTDSASTD

| | | Peptide sequence |
|---|---|---|
| #1 | 1) | To the different PP2Cα protein and mRNAs. |
| | 2) | To rattus norvegian neurona. pentroxin precursor mRNA. |
| | 3) | To Xenopus transcription factor IIIA. |
| | 4) | To Human DNA/RNA binding protein mRNA. |
| | 5) | To Human transcription factor IIIA. |
| | | and to other additional proteins to a lesser extent. |
| | | Homologues on the RNA level |
| #2 | 1) | PP2Cα mRNAs |
| | 2) | Human mRNA homologue to Xenopus transcription factor IIIA. |
| | 3) | Human DNA/RNA binding protein mRNA. |
| | 4) | Human transcription factor IIIA. |
| #3 | 1) | Different forms of PP2Cα |
| | 2) | C. elegans cosmid coding for DNA directed RNA |
| | | polymerase sigma chain. |
| | 3) | Potato mRNA for pyruvate kinase. |
| | 4) | C. elegans cosmid. |
| | 5) | Ictahurid herpes virus |
| | | DNA polymerase helicase |
| | 6) | A. thabana mRNA for UIsnRNA specific protein UIA. |
| | 7) | Ascaris lumbricoides small nuclear |
| | | RNA (snRNA UI-1 UI-2 HI-3 U-I genes). |

### References

Aladjem and Lavi, 1992. The mechanism of carcinogen-induced DNA amplification: In-vivo and in-vitro studies. Mutation Res. 276:339-344.

Atchison et al., 1965. Adenovirus-associated defective virus particles. Science 149:754-756.

Atherton-Fessler et al. (1993) Reversible tyrosine phosphorylation and cell cycle control. Semin. Cell. Biol., 4(6) :433-42.

Bantel-Schaal and Zur Hausen (1988a) Adeno-associated viruses inhibit SV40 DNA amplification and replication of herpes simplex virus in SV40-transformed hamster cells. Virology, 164:64-74.

Bantel-Schaal and Zur Hausen (1988b) Dissociation of carcinogen-induced SV40-DNA-amplification and amplification of AAV DNA in a Chinese hamster cell line. Virology, 166:113-122.

Berns, 1990. Parvovirus replication. Microbiol. Rev. 54:316-332.

Brown, et al. (1996) A defect in nurturing in mice lacking the immediate early gene fosB. Cell, Vol 86, pp. 297-309.

Burke and Olson, 1991. Preparation of Clone Libraries in Yeast Artificial-Chromosome Vectors” in Methods in Enzymology, Vol. 194, “Guide to Yeast Genetics and Molecular Biology”, eds. C. Guthrie and G. Fink, Academic Press, Inc., Chap. 17, pp. 251-270.

Burstyn, 1993. Suppression of SV40 DNA amplification by Adeno Associated virus. M.Sc. Thesis. Tel Aviv University.

Capecchi, 1989. Altering the genome by homologous recombination. Science 244:1288-1292.

Casto and Goodheart, 1972. Inhibition of adenovirus transformation in vitro by AAV-1. Proc. Soc. Exp. Biol. Med. 140:72-78.

Cech (1986) “RNA as an Enzyme”, Scientific American, 255:64-75

Cech (1990) “Self-Splicing of Group I Introns”, Annu. Rev. Biochem. 59:543-568

Chambers and Dahmus, 1994. Purification and characterization of a phosphatase from HeLa cells which dephophorylates the c-terminal domain of RNA polymerase II. J. Biol. Chem. 269(42):26243-26248.

Chambers et al. (1995) The activity of COOH-terminal domain phosphatase is regulated by a docking site on RNA polymerase II and by the general transcription factors IIF and IIB. Journal of Biological Chemistry, 270(25):14962-14969.

Cheung et al, 1980. Integration of the Adeno-associated virus gnome into cellular DNA in latently infected human Detroit 6 cells. J. Virol. 33:739-748.

Cohen, 1989. The structure and regulation of protein phosphatases. Annu. Rev. Biochem. 58:453-508.

Cohen, P. 1991. classification of protein-serine/threonine phosphatases: identification and quantitation in cell extracts. Methods. Enzymol. 201:389-399.

Cukor, et al, 1984. Biology of adeno-associated virus. In: Berns, K. I. (Ed.) The Parvoviruses. Plenum Press, New York, pp. 33-66.

Davies et al., 1992. Targeted alterations in yeast artificial chromosomes for inter-species gene transfer. Nucleic Acids Research, 20(11):2693-2698.

Dickinson et al., 1993. High frequency gene targeting using insertional vectors”, Human Molecular Genetics, 2(8):1299-1302.

de la Maza and Carter, 1981. Inhibition of Adenovirus oncogenicity by Adeno-associated virus DNA. J. Natl. Cancer Inst. 67:1323-1326.

Eden and Cedar (1994) Role of DNA methylation in the regulation of transcription. Curr. Opin. Genet. Dev., 4(2): 255-9.

Fodor et al, 1993. Multiplexed biochemical assays with biological chips, Nature 364:555-556.

Fukunaga, et al, 1993. Dephosphorylation of autophosphorylated Ca2+/calmodulin-dependent protein kinase II by protein phosphatase 2C. J. Biol. Chem. 268:133-137.

Gavrieli, et al, 1992. Identification of programmed cell death in situ via specific labeling of nuclear DNA fragmentation. J. Cell. Biol. 119:439-501.

Georg-Fries, et al, 1984. Analysis of proteins, helper dependence, and seroepidemiology of a new human parvovirus. Virology 134:64-71.

Gilboa, et al, 1986. Transfer and expression of cloned genes using retroviral vectors. BioTechniques 4(6):504-512.

Gossen and Bujard, 1992. Proc. Natl. Acad. Sci. USA 89, 5547-5551.

Hampel et al. (1993) “The Hairpin Ribozyme”, Methods: A Companion to Methods in Enzymology 5:37-42

Hermonat, 1989. The Adeno-associated virus rep78 gene inhibits cellular transformation induced by Bovine Papillomavirus. Virology. 172:253-261.

Hermonat, 1994. Down regulation of the human c-fos and c-myc proto-oncogene promoters by Adeno-associated virus rep78. Cancer Lett. 81:129-136.

Huxley et al., 1991. The human HPRT gene on a yeast artificial chromosome is functional when transferred to mouse cells by cell fusion. Genomics, 9:742-750 (1991).

Inouye, 1988. Antisense RNA: its functons and applications in gene regulation—a review. Gene. 72:25-34.

Jakobovits et al., 1993. Germ-line transmission and expression of a human-derived yeast artificial chromosome, Nature, 362:255-261.

Justement et al. (1994) Regulation of B-cell activation by CD45: a question of mechanism. Immunol. Today, 15(9):399-406.

Kafri, et al, 1992. Developmental pattern of gene-specific DNA methylation in mouse embryo and germ line. Genes Dev. 6:705-714.

Katz and Carter, 1986. Effect of adeno-associated virus on transformation of NIH 3T3 cells by ras gene and on tumorigenicity of an NIH 3T3 transformed cell line.

Kawasaki, 1990. Amplification of RNA. In: PCR protocols: A Guide to Methods and Applications, Innis M A, Gelfand D H, Sninsky J J, White T J, eds. Academic Press, pp21-27.

Kirschtein, et al, 1968. Inhibition of adenovirus 12 oncogenicity by adeno-associated virus. Proc. Soc. Exp. Biol. Med. 128:670-673.

Kleinschmidt et al. (1995) Sequence elements of the adeno-associated virus rep gene required for suppression of herpes-simplex-virus-induced DNA amplification. Virology, 206:254-262.

Lamb et al., 1993. Introduction and expression of the 400 kilobase precursor amyloid protein gene in transgenic mice”, Nature Genetics, 5:22-29.

Lau and Baylink (1993) Phosphotyrosy: protein phosphatases: potential regulators of cell proliferation and differentiation. Crit. Rev. Oncog., 4(4):451-71.

Lavi, 1981. Carcinogen mediated amplification of viral DNA sequences in SV40 transformed Chinese hamster embryo cells. Proc. Natl. Acad. Sci. USA 78:6144-6148.

Leonard and Berns, 1994. Adeno-associated viruses type 2: a latent life cycle. Proc. Natl. Acad. Sci. USA. 48:29-52.

Liang and Pardee, 1992. Differential display of eukaryotic messenger RNA by means of the polymerase chain reaction. Science 257:967-971.

Lichter, et al., 1990. High-resolution mapping of human chromosome 11 by in situ hybridization with cosmid clones. Science 247:64-69.

Mann et al., 1992. Mammalian protein serine/threonine phosphatase 2C: cDNA cloning and comparative analysis of amino acid sequences. Biochim. Biophys. Acta 1130:100-104.

Martin-Gallardo et al. (1992) Automated DNA sequencing and analysis of 106 kilobases from human chromosome 19q13.3. Nature Genetics, Vol. 1, pp. 34-39.

Mayor, et al, 1973. Influence of adeno-associated satellite virus on adenovirus-induced tumors in hamsters. Nature New Biol. 261:44-46.

Mayor, et al, 1976. Antibodies to adeno-associated satellite virus and herpes simplex in sera from cancer patients and normal adults. Am. J. Obstet. Gynecol. 126:100-104.

Martin-Gallardo, et al, 1992. Automated DNA sequencing and analysis of 106 kilobases from human chromosome 19q13.3. Nature Genetics. 1:34-39.

Mcclelliand, et al, 1995. RNA fingerprinting and differential display using arbitrarily primed PCR. TIG. 11:242-246.

McGowan and Cohen (1987) Identification of two isoenzymes of protein phosphatase 2C in both rabbit skeletal muscle and liver. Eur. J. Biochem., 166:713-722.

McGowan and Cohen, 1988. Protein phosphatase-2C from rabbit skeletal muscle and liver: an Mg 2+ dependent enzyme. Methods Enz. 159:416-426.

Nishikawa et al. (1995) Up-regulation of protein serine/threonine phosphatase type 2C during 1 alpha, 25-dihydroxyvitamin D3-induced monocytic differentiation of leukemic HL-60 cells. FEBS Lett., 375:299-303.

Ohishi, S. et al., Biochem. Intl. 28:345-351, 1992

Orita M, et al. Detection of polymorphisms of human DNA by gel electrophoresis as single-strand conformation polymorphisms. Proc Natl Acad Sci USA 1989; 86:2766-2770

Ostrove, et al, 1981. Inhibition of adenovirus-transformed cell oncogenicity by adeno-associated virus. Virology 113:521-533.

Pease et al., 1994. Light-generated oligonucleotide arrays for rapid DNA sequence analysis. Proc. Natl. Acad. Sci. USA 91(11):5022-5026.

Robinson, et al, 1994. TPD1 of Saccharomyces cerevisiae encodes a protein phosphatase 2C-like activity implicated in tRNA splicing and cell separation. Mol. Cell. Biol. 14:3634-3645.

Rommelaere and Tattersall, 1990. Tijssen P. (ed.), Handbook of Parvoviruses. CRC, Boca Raton, pp. 41-85.

Rothstein, 1991. “Targeting, disruption, replacement, and allele rescue: integrative DNA transformation in yeast” in Methods in Enzymology, Vol. 194, “Guide to Yeast Genetics and

Molecular Biology”, eds. C. Guthrie and G. Fink, Academic Press, Inc., Chap. 19, pp. 281-301.

Roth et al., 1990. Yeast alpha 2 repressor positions nuclesdomes in TRP1/ARS1 chromatin. Mol. Cell. Biol., 10:2247-2260.

Saadat, et al. (1994) Gene expression of protein phosphatases in rat ascites hepatoma cell lines. Cancer Detection and Prevention, 18(2):115-122.

Sato and Mayor, 1979. Adenovirus-associated virus polypeptides synthesized in cells coinfected with either adenovirus or herpesvirus. Virology 93:237-245.

Schedl et al., 1993. A yeast artificial chromosome covering the tyrosinase gene confers copy number-dependent expression in transgenic mice, Nature, 362:258-261.

Schlehofer et al. (1983) Inhibition of initiator-induced SV40 gene amplification in SV40-transformed Chinese hamster cells by infection with a defective parvovirus. Int. J. Cancer, 32:591-595.

Schlehofer (1994) The tumor suppressive properties of adeno-associated viruses. Mutation Research, 305:303-313.

Schlehofer, et al, 1986. Vaccinia virus, herpes simplex virus, and carcinogens induce DNA amplification in human cell line and support replication of helpervirus dependent parvovirus. Virology. 152:110-117.

Schlehofer and zur Hausen, 1982. Induction of mutations within the host cell genome by partially inactivated herpes simplex virus type 1. Virology 122:471-475.

Shiozaki and Russell (1995a) Counteractive roles of protein phosphatase 2C (PP2C) and a MAP kinase kinase homolog in the osmoregulation of fission yeast. The EMBO Journal, Vol. 14, No. 3, pp. 492-502.

Shiozaki and Russell (1995b) Cell-cycle control linked to extracellular environment by MAP kinase pathway in fission yeast. Nature, Vol. 378, pp. 739-743.

Shiozaki, et al, 1994. Protein phosphatase 2C, encoded by ptc1+, is important in the heat shock response of Schizosaccharomyces pombe. Mol. Cell. Biol. 14:3742-3751.

Siegl, et al, 1985. Characteristics and taxonomy of Parvoviridae. Intervirology 23:61-73.

Soderling (1993) Protein kinases and phosphatases: regulation by autoinhibitory domains. Biotechnol. Appl. Biochem., 19(Pt.2):185-200.

Sotomayor, et al, 1991. Role of the tumor derived cytokines on the immune system of mice bearing a mammary adenocarcinoma. J. Immunol. 147:2816-2823.

Sprecher-Goldberger, et al, 1971. Complement-fixation antibodies to adeno-associated viruses, aenoviruses, cytomegaloviruses and herpes simplex viruses in patients with tumors and in control individuals. Am. J. Epidemiol. 24:351-358.

Sullivan (1994) “Development of Ribozymes for Gene Therapy”, J. Investigative Dermatology (Suppl) 103:95S

Strauss et al., 1993. Germ line transmission of a yeast artificial chromosome spanning the murine α₁ (I) collagen locus, Science, 259:1904-1907.

Tamura et al. (1989) Molecular cloning of rat type 2C (IA) protein phosphatase mRNA. Proc. Natl. Acad. Sci. USA, Vol. 86, pp. 1796-1800.

Taylor, 1980. J. Histochem. Cytochem. 28:1021.

Tratschin, 1985. Adeno associated virus vector for high frequency of integration, expression, and rescue of genes in mammalian cells. Mol. Cell. Biol. 5:3251-3260.

Vindelov, et al, 1983. A detergent-trypsin method for the preparation of nuclei for flow cytometric DNA analysis. Cytometry 3:323-327.

Walz and Schlehofer 1992. Modification of some biological properties of Hela cells containing Adeno associated virus DNA integrated into chromosome 17. J. Virol. 66:2990-3002.

Wang et al. (1996) A Mg(2+)-dependent, Ca(2+)-inhibitable serine/threonine protein phosphatase from bovine brain. The Journal of Biological Chemistry, Vol. 270. No. 43, pp. 25607-25612.

Weinberg, R. (1996) E2F and cell proliferation: a world turned upside down. Cell, Vol. 85, pp 457-458.

Wenk and Mieskes (1995) Cytosolic and nuclear localization of protein phosphatase 2Cβ1 in COS and BHK cells. Eur. J. Cell Biology, 68:377-386.

Wera and Hemmings (1995) Serine/threonine protein phosphatases. Biochem. J., 311:17-29.

Wijsman, et al, 1993. A new method to detect apoptosis in paraffin sections: in-situ end-labeling of fragmented DNA. J. Histochem. Cytochem. 41:7-12.

Winocour, et al, 1992. Modulation of the cellular phenotype by integrated Adeno-associated virus. Virology. 190:316-329.

Yakobson, et al, 1989. Replication of Adeno-associated virus in cells irradiated with UV light at 254 nm. J. Virol. 63:1023-1030.

Yang et al. (1995) Inhibition of cellular and SV40 DNA replication by the adeno-associated virus rep proteins. Virology, 207:246-250.

Yakura (1994) The role of protein tyrosine phosphatases in lymphocyte activation and differentiation. Crit. Rev. Immunol., 14(3-4):311-36.

Young and Mayor, 1979a. Adeno-associated virus—an extreme state of viral defectiveness. Prog. Med. Virol. 25:113-132.

Young and Mayor, 1979b. Studies on the defectiveness of adeno-associated virus (AAV). 1. Effects of phosphonoacetic acid and 2-deoxy-D-glucose on the replication of AAV. Virology 94:323-341.

What is claimed is:

1. A method of detecting cancerous cells in a patient by detecting alterations of PP2Cα gene activity in a specimen isolated from the patient.

2. The method of claim 1 wherein the specimen is selected from the group consisting of tissue biopsies and bodily fluids.

3. The method of claim 1 further characterized by the alteration being a reduction in PP2Cα gene activity compared to normal controls.

4. The method of claim 1 wherein said detecting steps is further defined as assaying the specimen for mRNA complementary to PP2Cα DNA including polymorphisms thereof with an assay selected from the group consisting of in situ hybridization, Northern blotting and reverse transcriptase—polymerase chain reaction.

5. The method of claim 1 wherein said detecting step is further defined as assaying the specimen for a PP2Cα gene product including polymorphisms and peptide fragments thereof with an assay selected from the group consisting immunohistochemical and immunocytochemical staining, ELISA, RIA, immunoblots, immunoprecipitation, Western blotting, functional assays and protein truncation test.

6. The method of claim 5 wherein the specimen is bodily fluids selected from the group consisting of urine, blood, cerebralspinal fluid and saliva.

7. The method of claim 1 wherein the detecting of PP2Cα gene activity in a specimen is by determining alterations in phosphorylation patterns of proteins affected by the PP2Cα gene product.

8. A kit for detecting PP2Cα activity as set forth in claim 4, said kit comprising:
a molecular probe complementary to genetic sequences of a mRNA for PP2Cα including polymorphisms thereof and
detection means for detecting hybridization of said molecular probe and the mRNA thereby indicating the activity of the PP2Cα gene.

9. A kit for detecting a gene product associated with PP2C gene activity as set forth in claim 5, said kit comprising:
an antibody which with high specificity recognizes markers selected from the group consisting of the PP2Cα gene product including polymorphisms thereof and peptide fragments thereof, and
detection means for detecting the binding of the antibody thereby indicating the presence of the gene product.

10. A kit for detecting a gene product associated with PP2C gene activity as set forth in claim 5, said kit comprising:
an agent which mimics natural proteins which bind to the PP2Cα gene product including polymorphisms thereof and peptide fragments thereof, and
detection means for detecting the binding of the agent thereby indicating the presence of the gene product.

11. A non-human transgenic mammal or cell line containing an expressible nucleic acid sequence for human PP2Cα including polymorphisms thereof.

12. A non-human eucaryotic organism in which the equivalent genomic nucleic acid sequence for PP2Cα is knocked-out.

13. A vector comprising an expression control sequence operatively linked to the nucleic acid sequence of PP2Cα.

14. A host cell transformed with the vector of claim 13.

15. A vector comprising an antisence sequence of PP2Cα.

16. An antibody which specifically binds to an epitope of a gene product of PP2Cα including polymorphisms thereof which distinguishes the gene product of PP2Cα from the gene product of PP2Cβ.

17. An antibody of claim 16 conjugated to a detectable moiety.

18. An antibody of claim 16 selected from the group consisting of monoclonal and polyclonal antibody.

19. A polyclonal antibody of claim 18 raised against recombinantly produced PP2Cα.

20. A polyclonal antibody of claim 18 raised against the carboxy terminal peptide of pp2cα selected from the group consisting of NDDTDSASTD (SEQ ID No:1) and YKNDDTDSTSTDDMW (SEQ ID No:2).

21. A monoclonal antibody of claim 18 which does not cross-react with pp2cβ and which is raised against peptides selected from the group consisting of recombinantly produced pp2cα, NDDTDSASTD (SEQ ID No:1) and YKNDDTDSTSTDDMW (SEQ ID No:2).

22. A monoclonal antibody of claim 21 designated as 2A3.

23. An isolated and purified peptide selected from the group consisting of NDDTDSASTD (SEQ ID No:1), YKNDDTDSTSTDDMW (SEQ ID No:2) and PNKDNDGGA (SEQ ID No:3).

24. The peptide of claim 23 produced recombinantly.

25. A method of treating cancer including the steps of
a. determining the type of cancer and cells expressing the cancer,
b. preparing a vector which will specifically target the cancer cells including regulatory elements to control the expressibility of PP2Cα, and
c. administering the vector to the patient.

26. The method as set forth in claim 25 wherein the vector includes an AAV modifed sequence or part of the AAV sequence.

27. The method as set forth in claim 25 wherein the vector contains the CHINT sequences.

28. The method as set forth in claim 25 wherein the vector includes the silencer region (SEQ ID No:13).

29. The method as set forth in claim 25 wherein the vector includes the mini-silencer region (SEQ ID No:14).

30. A method of treating cancer including the steps of
a. determining the type of cancer and cells expressing the cancer,
b. preparing an antisense vector which will specifically target the cancer cells to control the expressibility of PP2Cα, and
c. administering the vector to the patient.

31. A pharmaceutical composition consisting of a vector and a pharmaceutically suitable carrier wherein the vector is selected from the group consisting of a vector which will specifically target the cancer cells and including regulatory elements to control the expressibility of PP2Cα and an antisense vector which will specifically target the cancer cells to control the expressibility of PP2Cα.

32. A method of treating diseases due to aberrant phosphorylation due to alteration of expression of PP2Cα including
a. preparing an antisense vector which will specifically target cells expressing aberrant phosphorylation to control the expressibility of PP2Cα, and
b. administering the vector to the patient.

33. A method of suppressing gene amplification by interrupting unscheduled interactions of DNA polymerase α primase with the gene product of PP2Cα by preparing an antisense vector which will specifically target the binding region of DNA polymerase a primase to the PP2Cα gene product and delivering the vector to the cells.

34. A method for the activation of the gene product of PP2Cα expressed on the surface of a cell to induce signal transduction.

35. The method of claim 34 wherein an antibody is used to bind to the gene product of PP2Cα.

36. A method of detecting cancer in a patient by detecting altered PP2Cβ gene activity in a specimen isolated from the patient.

37. The method of claim 36 further characterized by detecting an increase in PP2Cβ activity.

38. The method of claim 36 wherein the detecting of PP2Cβ activity is by assaying the specimen for mRNA complementary to PP2Cβ DNA including polymorphisms thereof with an assay selected from the group consisting of in situ hybridization, Northern blotting and reverse transcriptase—polymerase chain reaction.

39. The method of claim 36 wherein the detecting of PP2Cβ activity is by assaying the specimen for a PP2Cβ gene product including polymorphisms thereof with an assay selected from the group consisting immunohistochemical and immunocytochemical staining, ELISA, RIA, immunoblots, immunoprecipitation, Western blotting, functional assays and protein truncation test.

40. An antibody which specifically binds to an epitope of a gene product of PP2Cβ including polymorphisms thereof which distinguishes the gene product of PP2Cα from the gene product of PP2Cβ.

41. An antibody of claim 40 conjugated to a detectable moiety.

42. An antibody of claim 40 selected from the group consisting of monoclonal and polyclonal antibody.

43. A polyclonal antibody of claim 40 raised against recombinantly produced PP2Cβ.

44. A polyclonal antibody of claim 40 raised against the carboxy terminal peptide PNKDNDGGA (SEQ ID No:3).


SEQUENCE LISTING (20 sequences)

SEQ ID NO: 1 - protein, 10 aa, RAT
NDDTDSASTD

SEQ ID NO: 2 - protein, 15 aa, HUMAN
YKNDDTDSTSTDDMW

SEQ ID NO: 3 - protein, 9 aa, RAT
PNKDNDGGA

SEQ ID NO: 4 - DNA, 20 bp, ARTIFICIAL SEQUENCE
  PP2CALPHA cDNA PRIMER
aggatcaagtcataatggga

SEQ ID NO: 5 - DNA, 20 bp, ARTIFICIAL SEQUENCE
  PP2CALPHA cDNA PRIMER
gctggagtctgatttacaac

SEQ ID NO: 6 - DNA, 18 bp, ARTIFICIAL SEQUENCE
  PP2CALPHA cDNA PRIMER
gaagtagtcgacacctgt

SEQ ID NO: 7 - DNA, 21 bp, ARTIFICIAL SEQUENCE
  PP2CALPHA cDNA PRIMER
gtttgagaccttcaacacccc

SEQ ID NO: 8 - DNA, 23 bp, ARTIFICIAL SEQUENCE
  PP2CALPHA cDNA PRIMER
gtggccatctcttgctcgaagtc

SEQ ID NO: 9 - protein, 6 aa, RAT
MGAFLD

SEQ ID NO: 10 - DNA, 28 bp, ARTIFICIAL SEQUENCE
  PP2CALPHA cDNA PRIMER
cgggatccgcatgggagcatttttagac

SEQ ID NO: 11 - protein, 5 aa, RAT
TDDMW

SEQ ID NO: 12 - DNA, 27 bp, ARTIFICIAL SEQUENCE
  PP2CALPHA cDNA PRIMER
cgcggatccttaccacatatcatcagt

SEQ ID NO: 13 - DNA, 64 bp, ARTIFICIAL SEQUENCE
  SV40 SILENCER REGION
actccatcactaggggttcctggaggggtggagtcgtgacgtgaattacgtcatagggttaggg

SEQ ID NO: 14 - DNA, 22 bp, ARTIFICIAL SEQUENCE
  SV40 MINI-SILENCER REGION
actcccatcactaggggttcct

SEQ ID NO: 15 - DNA, 1573 bp, RAT
  misc_feature (136)..(1562): N = ANY NUCLEOTIDE
aagcttgtcaaaattactattcagtgtgatttttagtggatgaaacctcatgactagtatattatgacattagctttgcgtagtgaaggcacaagctgctaagtggttagggatgtattttgccgtagcctgtatcacnccaggtcctgggctcggttcctagcattacaggaaaaagcaggcggtggttgacctttaatgaatggatttttcaatttagaagttggtttcattttaaagaattcaaaaatgttccccatagcactttgttttgacattgagatcagctgctaattgaggtccagtatatacttagaaaactgagcgaaactttgatggacacacacacacacccctgttgttcatttaataattgaactaaataaaatactgtttagtcatccacgtaagcaagaggcctgtgtaaacagtatttgtattagtaaaaactttataacatagttacataatcagcatcattttttttatggaccttatagttggctacttcactgggtttgttataatttaatcagactcctaaataggttaaatttctgaattgcctacttcagttttgaagaattattttgtttcataatttcccatgcatatctggtaaataattctggattgtttctaaaggggagagcaaggtctcttatgcaaagtgaaaatctagatatgctgtttgtaagaatataatagtgataaagtagtgtccttttgctcagtgcctccattcttaccaggctgtgactgatcttcagtattattcagacagtcactattaatatatccgttgcacagtggggaaattgagggaagttagataggcatcgggtatcttaatcataactcacatatacccagctggctagtcagcctagctaagacagttcacacccagttgaggcagcttgctgttggccattagtaggtaacttaatggcttggtttcttcactggtaaggtggggatataataatgccaataattgcataatgattaaagacattaatatattccataaaatttcctgaatagtgcttagctggtacccctccccacacatgcaccccagtccaatgttcagatgtttactttgttaagcccagttaatccattccccctaatatcttctcccagtttgaagaangttgaagaatgttgggcttgttagtttaattttttaagaagcatatcatgttgcttttttaaaacatgtttctttgggttttggcttccccttttggaaagaattccaatttacacttatggaagaaagccattgtcccctccaatttccccccctgtccctttccaatacagcccaactccccatgttttgacttcctcccctgaaccaccccgttctcctgtttttccctcccccanaaaaaaaacccaataatttgactttggtaattgaatttcccgccngttaggcncctgaattgccgaaataattcccccgtgcncccngganttttggcaccccctgccccttaacctgttctgctgccccccatttttaaatggcttgccgcnttacnccaaanactgcctttcc

SEQ ID NO: 16 - DNA, 2580 bp, RAT
  misc_feature (568)..(2580): N = ANY NUCLEOTIDE
ctcgatctcacaaagtcacagagctcttcgtttcccatgacatcccagataccatcacatgcaagaataatgaactgatcgtcctcttcagacctttcaatatcatggacttctggctctggtgagacgagctgctctgtgggaccttttccatggacacatttgtaatcgaaatccccaagggcccttgacacagccagagagccatttacacgctgaatcatcacagagccccctgcattctgaattcgttctttttccagcgggttacttggtttgtggtcttgtgtgaagaagtgaactttcctgtttctacaaagcaaacctctcgagtctccacagttaatgaagtaagtatgtttggggagaaattaagacccccacagctgtttgacccacttcctatctgcaccatgttttccttcctcctgacatgactcctcatgttgtttccatcaatctcccagaaaaacctgttcctgatccccattcctttacattttcccacagaaaggtgctccctgcagagccttttaaaatccctggtttattggtgatgttgattctnaacaaatgctccacagccagtatttnggcaaccttgaaaaaccagcatgcccatccatatacagccaagaatgaccatgttctccagttccactttnggcaaacccaatccacagccgttntgcgcatcctcccatttcaactccgcccaaccnttgcntgctgcnttaagccatatcgcaacccatcccccctgccccctggggcattatgcntttccatctttggttgtctaaaatgctcccattatgacttgatcctctaggtctgcaaaggaagagaaataagaaagttagtaactgtctttgaaacaaagcacacatccaacagtctttttgaagcacctacgagatacaaggaaacgtaaaaactcataggctatagccataagcattgttctactgacttggaaaatgtagagattaataagaaagggaaaggctgatcaagtacagctcaaccagacaagcagcagatggaactaagtcaccaggtaaaagagagcttgtttgcctctctgtgataccaaggaggcccagcagtgaccattaacttacatgaactaggcaagatttcagggtgcattcatcatatgtaacctctcaattaagttgtgtgttgattaaaaaaaataattcatagaaacatacaagtatctactacttcagggaaccttagctaagtactcaggaatgttgagagtttgattccatgctatttagttttgtttctacaactagatacctttggtaaaaataaaaagtaattactcacactggtccaaattttcagtgccttgtgcaggtcattctctttagctggaattccctgcctcacctctttaccaacagaaaaaaaatacacctgtttctatcctttgaaatccagttcaattgttcccccttcctccagactttacagtccttgaaaaaaacaagttattaactacagaagtcagcttccatttccagttnggaatgttttttaatgaacaattttattgttcnaaatctnacnatatgataactaancnaatggtaataatattttcanccctgccctatggccgctntttttaatcctnaaaaaaatcnaaggtctattccncccnnccttgccaatacttnacancnccagttccctgatctggaatggacccacaaaggtcaagacttaggttancccttgctcacaaactaaagaaaatcttaaaggagaacagaatactgaagagagaaatgagggtgaaggacagtgttcaggtgacgttctgaaaccaggggactaaanataccanaantggtgttncagacagaaatggtatggaaaactccttaggaaagaaatgacanntnttgtttcgcagcaacccccncacatggctttctctttttccttctgctgattaactgatgcacntggtanaaaagtcaacanacccctcctccacncagactcccaccgagtacannggcccatgtgctcantacactctgccccaaactcnnannattcattcnnctccccntgtnatttatnagggcctttcccntcagttntctnatcnccaacgganattanccttccannnatttacccccnntttgtacancacatnntggcnngtgccacngttangcgtcggcntccctgttncactncatccctcatcnttaggccangtttgattctccngtgcanantttccgcannancntaccccttgcaccntccatntctnnggaanaacctccggttctgaatctnccccnntcccgtcnctcccccnttctttcttttctctanttttttccnnggnacgggttgnggtnaatnaanncccctccttcgtctattcancccttcctatgnacacttcctgnccccctatctctctatntnctnctctctatatctnnatcccntcttcncntgccnctccctngtnttnnancgggtatttnttnttctcctcntcttcttcccctntntanccntnctncnnncnnnccc

SEQ ID NO: 17 - DNA, 830 bp, RAT
  misc_feature (528)..(822): N = ANY NUCLEOTIDE
tgggggagaggactgaaatatttccacagcctttttattggtggtgatggtagtgatggttaggattccttctttctttctttctttctttctttctttctttcttttttttttttttttttttttttttgagacagggtttctctgggtactcctggaactcactttgtggaccatgaatgacatgaatacttcgatatatacatacatacaaagacacatatttttaaaaagagaattagagtagagctggggcaattgtggaacacacctttaacctcaggcagatttctgcgttcaaggtcaccttggattacaaggcagctagggctacacagagaaaccatatctcaaaaaaaagaaaaaataatgaaagaaagaaaggaaggaaggaaggaaggaaggaaggaaggaaggaagaaaggaaggtaggaagaaaggtattttcctaaaaaaaaaaaaaaaaaaaatttattccgggcagtggtggcaaatgcttttaatcccaccatttgggaaagcagaggcagacagattaaattttcaaggcccacctggtcctacacagtgaattccaggaacacctaggtttacccanaaaaaacccccccttgaaataaacaaaaataaattaaataaataaaatttaaaaataaaacccgggcgttaaacccncttttatccccccacttnggaagcaaaagccggcngatttctgaattcnaggccnccctgtctatgaattanttcccngaacacccnaatttttcnaaaaaccccccntttcttaaaaaanccaaattattattnattaattaaatnaaattacc

SEQ ID NO: 18 - DNA, 838 bp, RAT
  misc_feature (578)..(837): N = ANY NUCLEOTIDE
ggagtccaacaatggtttccacttgtctggcggccgctctagagtttcccataagctggactgagagatggtgtgattgctgtgggtgacaaagacagaggcacctttcatctctacccttctcttgttttgttgtttgtttgagaccggttcccactatgtagaccaggctggaggacagggtctcactatgtagaccaggctggccttgaactcaaagacatctgcctgcctctgcctcctgagggctgggattaaaggcgtgtgctgccactgacagcttctatcctcctgtcatcagtcccggctcacagggccagaagatctcttctatgcttccactatttccccaatccattcccacggcagcctctccatctccctaccaccaagacagcagcctagtgatataacaaaacttttattcacaggaaaccggaaaacaaaatcacaaccaatcatttctatctagtccctgccctagccctccctccaagcccctacatatcctccatctgagggggatgcatgcgttgggtgggagctgccggcatccttatcctggttcctggagtagngaagagtggttcttttcaacgnctagggnnctcccctccaagttnggacctctcttcccaggncttcncccctccctnacagggnacaaaaaaccaggnacggcacnacgccaggnaggaagggactcttggnaatgttgggcaggacttgtcctcagaattccnnggaggaatcaagggccttgaattcgggaaccactnccgaggncttcancanggcanagttcaattttccatcccggttggcccancctggccng

SEQ ID NO: 19 - DNA, 180 bp, RAT
tagtgccggtcaaggaactgaacgtgcgattccgggacaggctacccactccgatcccaggagaagttgtcatggtgagggccaccctaggtctctgcccctgctgtgtcccccatcttacccatccagtaggatctagaggctgtcgcccccttgtggaatgcacagaagtcacaagcg

SEQ ID NO: 20 - DNA, 175 bp, HUMAN
cctcacctccgccctgtttcgtccaggtcctccgggtcaggctacccccgtcgccgccagagcgcgggggaggggagagcttcctttgtctcctatgcctcctccccccatcccggctctcctgcgggcaagcgccgaggggacaccggggagtaccccacctgaacctctgggg